(19) United States
(12) Patent Application Publication
(10) Pub. No.: US 2021/0369623 A1
(43) Pub. Date: Dec. 2, 2021

(54) COMPOSITION THAT FORMS LIQUID CRYSTALLINE PARTICLES

(71) Applicant: Zeenar Enterprises Pty Ltd, Brighton (AU)

(72) Inventors: David KANNAR, Melbourne (AU); Tomer MADMON, Melbourne (AU)

(21) Appl. No.: 17/288,310

(22) PCT Filed: Oct. 25, 2019
(86) PCT No.: PCT/AU2019/051180

§ 371 Date: Apr. 23, 2021

(30) Foreign Application Priority Data
Oct. 25, 2018 (AU) 2018904059

(51) Int. Cl. A61K 9/20; A61K 31/727

(52) U.S. Cl. CPC A61K 9/2072; A61K 31/727; A61K 9/2027; A61K 9/2013

(57) ABSTRACT

The invention provides an oral disintegrating tablet (ODT) for administration to oral mucosa comprising an amphiphilic compound capable of self-assembling into liquid crystalline particles when contacted with a hydrophilic solvent, a therapeutically effective amount of a 1 to 10 kDa active ingredient and a pharmaceutically acceptable disintegrant, wherein the 1 to 10 kDa active ingredient is orally bioavailable. The water channels of the liquid crystalline particle may be increased in size. ODTs with enhances water channels may also be useful for delivery of active ingredients of less than 1 kDa.

### FIELD OF THE INVENTION

The present invention relates to compositions capable of forming liquid crystalline particles, methods for the manufacture of said compositions and methods of medical treatment using said compositions. The compositions are optionally oral disintegrating tablets. In particular, the present invention relates to tablets formulated to deliver an active ingredient via the oral mucosa.

### BACKGROUND OF THE INVENTION

Liquid crystalline particles are of interest in drug delivery. They can prolong the release of some active ingredients and are being investigated as vehicles to deliver lipophilic drugs. Dosage forms using liquid crystalline particles still require significant development so that the delivery of various active ingredients can be appropriately controlled.

An orally disintegrating tablet or orally dissolving tablet (ODT) is a drug dosage form that is a solid oral preparation that disintegrates rapidly in the oral cavity. ODTs differ from traditional tablets in that they are designed to be dissolved in the oral cavity rather than be swallowed whole. For example, an ODT can dissolve on the tongue, sublingually (under the tongue) or buccally (ie on the cheek).

ODTs have been used for patients who experience dysphagia (difficulty in swallowing) and when it is convenient to have a tablet that can be taken without water. Often, these are dosage forms specially designed for children or the elderly.

Drugs delivered via the oral mucosa can have a faster onset of effects than drugs delivered by swallowed tablets or capsules. Glyceryl trinitrate (GTN) is a known drug that has been formulated as an ODT for rapid drug absorption via the oral mucosa because rapid absorption is critical to prevent a heart attack. Unfortunately, for many other active ingredients, rapid onset is unsuitable because the blood concentration of the drug would rise too fast or too far causing side effects and/or resulting in a short therapeutic effect.

Delivery via the oral mucosa is of interest for drugs that yield low bioavailability through the digestive tract but are inconvenient to administer parenterally, such as steroids and narcotic analgesics. In addition to rapid action, oral mucosal delivery offers significant advantages for some active ingredients by avoiding extensive first pass metabolism. This could mean less drug is required to produce an equivalent effect compared to oral administration. Associated side effects could also be reduced. Oral mucosal delivery is not currently used where slow release of a drug is required. If speed of oral mucosal absorption could be slowed or managed, then this delivery route could be useful for a wider range of active ingredients. It would be particularly useful if the absorption of the active ingredient could be slowed down but the dosage form designed so that it does not remain in the mouth for an extended period.

Dosage forms for oral mucosal delivery also currently have limited utility for lipophilic drugs, which have difficulty absorbing across the mucosal membrane. It would be useful to have a dosage form that assists with delivery of lipophilic drugs via oral mucosa.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

ODTs with Large APIs

The inventors of the present invention have developed an ODT that contains an amphiphile, which forms liquid crystalline particles when the ODT contacts a hydrophilic fluid. The ODT was originally developed to deliver small molecules via the oral mucosa (see International patent application no PCT/AU2018/050366). Key researchers in the field have expressed an expectation that larger active pharmaceutical ingredients (APIs) would not release from a liquid crystalline phase and this opinion had been supported by some data. Consequently, it was expected that the ODT technology could not be directly applied to use with larger active pharmaceutical ingredients (APIs). Due to their size, there could be difficulty with capturing the large APIs in the liquid crystalline particle and/or, if captured, the large APIs could have difficulty diffusing out of the liquid crystalline particle. Previous studies have shown that larger macromolecules have too slow or non-existent release, meaning delivery of such macromolecules using liquid crystalline particles did not look promising.

Surprisingly, the inventors of the present invention have prepared an ODT comprising macromolecules of 1 to 10 kDa that forms liquid crystalline particles when contacted by a hydrophilic solvent. The ODT also releases, rather than sequestering, the 1 to 10 kDa API making the ODT a suitable API delivery vehicle.

In a first aspect, the present invention provides an oral disintegrating tablet (ODT) for administration to oral mucosa comprising an amphiphilic compound capable of self-assembling into liquid crystalline particles when contacted with a hydrophilic solvent, a therapeutically effective amount of a 1 to 10 kDa active ingredient and a pharmaceutically acceptable disintegrant, wherein the 1 to 10 kDa active ingredient is bioavailable. Bioavailability for this transmucosal dosage form can be different to oral bioavailability for other oral dosage forms. The presence of the self-assembled structures allows for the oral delivery of active ingredients that are not orally bioavailable in other environments. It will be evident to the skilled person that bioavailability for the transmucosal dosage form of the invention requires that the active ingredient not be sequestered in the liquid crystalline particles formed when the ODT contacts saliva but that where the active ingredient is encapsulated in the liquid crystalline particles it is released from those particles with suitable speed. Where administration is by oral mucosal delivery, the dosage form optionally assists the large API across the oral mucosa. Bioavailability testing options are described further in the detailed description.

Alternatively, the first aspect of the invention provides, an oral disintegrating tablet (ODT) suitable for administration to oral mucosa comprising an amphiphilic compound capable of self-assembling into liquid crystalline particles when contacted with a hydrophilic solvent, a therapeutically effective amount of a 1 to 10 kDa active ingredient and a pharmaceutically acceptable disintegrant.

Alternatively, the first aspect of the invention provides, an oral disintegrating tablet (ODT) suitable for administration to oral mucosa comprising an amphiphilic compound capable of self-assembling into liquid crystalline particles when contacted with a hydrophilic solvent, a therapeutically effective amount of a 1 to 10 kDa active ingredient and a pharmaceutically acceptable disintegrant, wherein the ODT facilitates systemic administration of the active ingredient across the oral mucosa.

Optionally, the active ingredient in the first aspect of the invention is about 1 to about 9 kDa, about 1 to about 8 kDa, about 2 to about 8 kDa, about 2 to about 7 kDa or about 2 to about 6 kDa. Alternatively, the active ingredient is 1.3 to 10 kDa, 1.5 to 10 kDa, 1.3 to 9 kDa or 1.5 to 9 kDa. In a preferred embodiment, the active ingredient is about 4 to about 5 kDa.

Optionally, (i) the amphiphile forms liquid crystalline particles at physiological pH, temperature and/or salinity; and/or (ii) the hydrophilic solvent has physiological pH, temperature and salinity.

Compositions, Self-Assembled Structures and ODTs with Water Channel Enhancers

Liquid crystalline particles have water channels in addition to hydrophobic regions. The water channels are where hydrophilic active ingredients are thought to be held and diffuse from. The water channels are a certain size in certain conditions for a certain amphiphile, such as glycerol monooleate. The size of the water channels is thought to limit the amount of active ingredient and the size of active ingredients that can be held within the liquid crystalline phase. Increasing the size of the water channels could potentially increase the speed of dissolution for the active ingredient, allow for greater loading of active ingredient and/or allow for loading of larger active ingredients. These effects are expected, in particular, for hydrophilic active ingredients, which are expected to be contained in the water channels in the liquid crystalline particles. For the liquid crystalline phase to be useful in drug delivery, the active ingredient needs to load into the liquid crystalline particles and release out of the liquid crystalline particles at a suitable speed. The very slow release of large active ingredients from liquid crystalline phase limits the utility of liquid crystalline phases in the delivery of larger active ingredients.

The inventors of the present invention have developed ways to increase the size of the water channels in the liquid crystalline particles to increase the loading and/or release of hydrophilic active ingredients including enabling delivery of larger hydrophilic active ingredients.

This allows the use of ODT formulations for drugs previously not suitable for that dosage form, while still providing patients with the convenience of a fast disintegrating ODT. The ODT developed also disintegrates rapidly without interfering with the formation of the liquid crystalline particles.

Therefore, in a second aspect, the present invention provides a composition comprising an amphiphile capable of self-assembling into liquid crystalline particles when contacted with a hydrophilic solvent, a therapeutically effective amount of an active ingredient and a water channel enhancer. Optionally, the water channel enhancer is one or more selected from the group consisting of rosuvastatin, oxycodone, ibuprophen lysine, Tween 80, sodium cyclamate, saccharin sodium, Poloxamer 188, Poloxamer 407, Mg Trisilicate, sodium lauryl sulphate (SLS), menthol, stearic acid, butylated hydroxyl toluene and sodium bicarbonate. Alternatively, the water channel enhancer is one or more selected from the group consisting of Tween 80, sodium cyclamate, saccharin sodium, Poloxamer 188, Poloxamer 407, Mg Trisilicate, sodium lauryl sulphate (SLS), menthol, stearic acid, butylated hydroxyl toluene and sodium bicarbonate. Alternatively, the water channel enhancer is one or more selected from the group consisting of Tween 80, sodium cyclamate, saccharin sodium, Poloxamer 188, Poloxamer 407, Mg Trisilicate, sodium lauryl sulphate (SLS), stearic acid, butylated hydroxyl toluene and sodium bicarbonate.

The water channel enhancer preferably has a secondary function in the dosage form. For example, the water channel enhancer may also be the active ingredient, a sweetening agent, taste masking agent, surfactant, penetration enhancer, solubilising agent, lubricant, glidant, antioxidant, preservative, flavouring agent, and/or alkalising agent. However, the water channel enhancer cannot be the same ingredient as the amphiphilic compound. Therefore, if the water channel enhancer is Tween 80 and/or SLS, the amphiphilic compound cannot be Tween 80.

In a third aspect, the present invention provides a self-assembled structure including liquid crystalline particles comprising an amphiphilic compound, a therapeutically effective amount of an active ingredient, a water channel enhancer and a hydrophilic solvent. The liquid crystalline particles are optionally in a liquid crystalline phase. The amphiphilic compound, active ingredient, water channel enhancer, hydrophilic solvent, ratios of ingredients and types of self-assembled structure are as described elsewhere in the specification. The water channel enhancers and their functions are optionally as described for the second aspect of the invention.

In a fourth aspect, the present invention provides an oral disintegrating tablet (ODT) for administration to oral mucosa comprising an amphiphilic compound capable of self-assembling into liquid crystalline particles when contacted with a hydrophilic solvent, a therapeutically effective amount of an active ingredient, a water channel enhancer and a pharmaceutically acceptable disintegrant. The water channel enhancers and their functions are optionally as described for the second aspect of the invention.

The water channel enhancer is an ingredient that increases the size of the water channels in the liquid crystalline structure formed by the amphiphile. The skilled person will understand that each amphiphile capable of self-assembling into a liquid crystalline phase will have a usual liquid crystalline particle structure with a usual water channel size when in a hydrophilic solvent such as water, saliva, phosphate buffered saline (PBS) or citric acid buffer at the pH of the mouth (about 5.5 to 7.4). For the purpose of the patent specification, the usual lattice and water channel sizes are those determined in PBS at pH 7.4 (unless an ingredient is incompatible with PBS and then citric acid buffer at pH 6.6 is used). Optionally, the water channel enhancer increases the size of the water channels of liquid crystalline structure within formed when the amphiphilic compound self-assembles by at least 5 (or at least 10) A (angstroms) when compared to their usual size.

The size of the water channels in bulk phase or a colloidal dispersion can be measured indirectly by determining the lattice size for the liquid crystalline particle. Conversion from lattice size to water channel size is explained below. Optionally, the water channel enhancer in the second to fourth aspects of the invention increases the lattice size of the liquid crystalline structure within formed when the amphiphilic compound self-assembles by at least about 10 nm, when compared to the usual size for the amphiphile. Optionally, the lattice size is increased by at least about 20 nm or at least about 30 nm. In some instances, the lattice size is increased so far that the liquid crystal structure is altered and converts a structure with larger water channels. For example, Pn3m cubic could convert to Im3m cubic.

By way of example, the usual lattice size for Pn3m cubic glycerol monooleate is about 88 to about 92 nm. Using a water channel enhancer increases the lattice size to at least 100 nm. The lattice size can optionally be increased to at least 110 nm or at least 120 nm. In some embodiments, the water channel enhancer causes the Pn3m cubic structure to convert to Im3m cubic structure.

Therefore, in some embodiments, the amphiphilic compound, in the presence of the water channel enhancer, is capable of self-assembly into cubic Im3m liquid crystalline particle and (ii) the amphiphilic compound in the absence of the water channel enhancer is capable of self-assembling into cubic Pn3m liquid crystalline particles.

There are three types of water channel enhancers. First, there are simple water channel enhancers ie those that increase the lattice/water channel size until the liquid crystalline form no longer holds. These do not facilitate a transition to an alternate liquid crystalline structure with inherently larger water channels. Second, there are complex water channel enhancers ie those that increase the lattice/water channel size until a certain point and then facilitate a transition to an alternate liquid crystalline structure with inherently larger water channels (for example a V2(Pn3m) to V2(Im3m) transition. Third, there are simple transition enhancers ie those that do not result in a significant increase in water channel size in a given liquid crystalline structure but do facilitate a change to an alternate liquid crystalline structure with inherently larger water channels. Examples of each type are provided below.

Optionally, the water channel enhancer in the second to fourth aspects of the invention is a simple water channel enhancer, a complex water channel enhancer and/or a simple transition enhancer.

In some embodiments of the second to fourth aspects of the invention, the active ingredient is the water channel enhancer, for example, where the active ingredient is rosuvastatin, oxycodone and ibuprofen lysine, the active ingredient can also be the water channel enhancer. Optionally, the water channel enhancer is selected from the group consisting of Tween 80, sodium cyclamate, saccharin sodium, Poloxamer 188, Poloxamer 407, Mg Trisilicate, SLS, menthol, stearic acid, butylated hydroxyl toluene and sodium bicarbonate. Optionally, the water channel enhancer is selected from the group consisting of sodium cyclamate, saccharin sodium, Poloxamer 188, Poloxamer 407, Mg Trisilicate, menthol, stearic acid, butylated hydroxyl toluene and sodium bicarbonate. Optionally, for ODTs of the invention, the water channel enhancer is Tween 80, SLS, rosuvastatin, sodium bicarbonate or combinations thereof.

Optionally, the water channel enhancer is present in an amount at least 5% or at least 10% of the combined weight of the water channel enhancer and the amphiphilic compound. Alternatively, the water channel enhancer is present in an amount at least 30% or at least 35% of the combined weight of the water channel enhancer and amphiphilic compound.

In some embodiments of the first aspect of the invention, the ODT further comprises one or more water channel enhancers as discussed above.

In the second to fourth aspects of the invention, the active ingredient is preferably bioavailable. Bioavailability for this transmucosal dosage form can be different to oral bioavailability for other oral dosage forms. The presence of the self-assembled structures enables the delivery of active ingredients that are not orally bioavailable in other environments. It will be evident to the skilled person that bioavailability requires that the active ingredient not be sequestered in the liquid crystalline particles formed when the ODT contacts saliva but that where the active ingredient is encapsulated in the liquid crystalline particles it is released from those particles with suitable speed. Bioavailability can be tested in vitro using the Ussing chamber testing set out in the examples.

### Embodiments of the ODTs of the Invention

In some embodiments of the ODTs of the invention (for example, in embodiments of the first and fourth aspects of invention), the disintegrant is about 1 to about 60% w/w of the tablet and the oral disintegrating tablet disintegrates in less than 2 minutes. Optionally, disintegration speed is measured in a basket-rack assembly with water at 37° C. as a solvent and in accordance with Appendix XII A. Disintegration of the European Pharmacopoiea edition 9.0 (Ph. Eur. Method 2.9.1). The amphiphile optionally also prolongs the release of the active ingredient. It is preferred that the ODT disintegrates in 1 to 110, 1 to 100, 1 to 90, 1 to 80, 1 to 70, 1 to 60, 1 to 50, 1 to 40, 1 to 30, 10 to 30, 20 to 60, 30 to 90, 1 to 20 or 1 to 10 seconds following contact with a hydrophilic solvent or administration to the oral cavity.

In some embodiments (for example, in embodiments of the first and fourth aspects of invention), when the ODT of the invention is administered to the oral mucosa, the ODT facilitates systemic administration of the active ingredient across the oral mucosa. The non-ODT compositions and self-assembled structures of the invention optionally also facilitates systemic administration of the active ingredient across the oral mucosa.

Preferred ODTs of the invention are stable for at least 2 years, at least 1 year, at least 9 months, at least 6 months or at least 3 months. Alternatively, the ODT is stable for at least 3 years. In some embodiments, the ODTs of the invention are stable for a pharmaceutically acceptable shelf-life. Optionally, both the active ingredient and the amphiphile are stable over the shelf-life such that there is (i) sufficient active ingredient remaining and (ii) sufficient amphiphile remaining to form liquid crystalline particles resulting in delivery of a therapeutically effective amount of the active ingredient at the end of the shelf-life.

In some embodiments ODTs are stable at 25° C./60% relative humidity for at least 2 years, at least 1 year, at least 9 months, at least 6 months or at least 3 months and/or stable at 5° C. for at least 2 years, at least 1 year, at least 9 months, at least 6 months or at least 3 months, in particular, preferred ODTs retain about 90% or about 95% or more active ingredient following storage at either 25° C./60% relative humidity or 5° C. for at least 2 years, at least 1 year, at least 9 months or at least 6 months. Alternatively, the ODT is stable at 25° C./60% relative humidity for at least 3 years. Optionally, the ODT retains at least 80%, 90% or 95% of the amphiphile following storage at either 25° C./60% relative humidity or 5° C. for at least 2 years, at least 1 year, at least 9 months or at least 6 months. Optionally, the ODT retains about 90% or about 95% or more active ingredient and at least 80%, 90% or 95% of the amphiphile following storage at either 25° C./60% relative humidity or 5° C. for at least 2 years, at least 1 year, at least 9 months or at least 6 months. Optionally, the active ingredient is a statin and there is also no change in lactone or 5-oxo-rosuvastatin calcium levels following storage at 5° C. for at least 3, 6 or 9 months and/or less than 0.5% w/w lactone and less than 0.4% w/w 5-oxo-rosuvastatin calcium following storage at 25° C./60% relative humidity for at least 3 or 6 months.

In some embodiments of the first and fourth aspects of the invention, the amphiphilic compound is optionally present at an amount of about 1 to 20% w/w of the ODT. In some embodiments the amount of amphiphilic compound is 3 to 10% w/w, 4 to 8% w/w, 4.5 to 7.5% w/w or 5 to 7% w/w. In preferred embodiments the amount of amphiphilic compound is about 5% w/w or about 7% w/w. Use of about 5% w/w of amphiphilic compound is preferred for formulations with an about 1:1 w/w ratio of amphiphilic compound to active ingredient. Use of about 7% w/w of amphiphilic compound is preferred for formulations with an about 4:1 w/w ratio of amphiphilic compound to active ingredient.

In preferred embodiments, the pharmaceutically acceptable disintegrant is present at an amount of about 1 to about 60% w/w of the ODT. In some embodiments the amount of pharmaceutically acceptable disintegrant is about 10 to about 50%, about 20 to about 60%, about 20 to about 50%, about 20 to about 40%, about 15 to about 40% w/w, about 20 to about 30% w/w, about 10 to about 20% w/w of the ODT. Alternatively, the ODT is prepared with low disintegrant content of about 1 to about 10% w/w of the ODT.

In some embodiments, the pharmaceutically acceptable disintegrant is selected from the group consisting of sodium starch glycolate, copovidone, crosslinked polyvinylpyrrolidone (crospovidone) or a derivative of crospovidone such as, crosslinked sodium carboxymethyl cellulose (croscarmellose sodium) sodium/calcium carboxymethylcellulose, sodium bicarbonate, microcrystalline cellulose, low-substituted hydroxypropylcellulose or sodium starch glycolate. It is possible to add crospovidone to the ODT independently or in the form of a blend such as Pharmaburst™, which contains 7-15% crospovidone.

Preferred formulations of the invention comprise two or more disintegrants. It is preferred if one of the two or more disintegrants is crospovidone. In some embodiments, the two or more disintegrants include both crospovidone and sorbitol copovidone. In some embodiments there are two disintegrants. These disintegrants are optionally crospovidone and copovidone or sodium starch glycolate and crospovidone.

Preferred formulations of the invention comprise three or more disintegrants. The three or more disintegrants are preferred to be crospovidone, copovidone and sodium starch glycolate.

Where the ODT includes crospovidone, the crospovidone is preferred to be about 5 to about 45%, about 10 to about 45% w/w of the formulation, about 15 to about 35% w/w of the formulation or about 20 to about 25% w/w of the formulation. Where the formulation includes crospovidone, and sodium starch glycolate, the preferred amounts of crospovidone are as above and the preferred amount of sodium starch glycolate is about 3 to about 8% w/w or about 5% w/w of the formulation.

In some embodiments of the ODTs of the invention, as the ODT disintegrates in the oral cavity, the amphiphilic compound and/or self-assembled structures form a mucoadhesive layer on the oral mucosa. Optionally, the mucoadhesive layer formed facilitates the delivery of the active ingredient across the oral mucosa.

Optionally, the ODT has a hardness of about 0.5 to about 6 kp, about 0.5 to about 4 kp, about 1 to about 4 kp or about 1 to about 3 kp, about 1 to about 2 kp or about 1 to about 1.5 kp. Tablet hardness or breaking force is determined according to chapter <1217> of the United States Pharmacopeia on tablet breaking force.

Optionally, the ODT is under 500 mg.

The ODT is optionally 5 to 15 mm or 8 to 12 mm in diameter.

Optionally, when the ODT is administered to the oral mucosa, the tablet disintegrates and the amphiphilic compound self-assembles into liquid crystalline particles.

Some early ODTs prepared by the inventors of the present invention (see, for example WO 2014/179845) were slow to disintegrate, for example, some formulations took 45 minutes or 30 minutes to disintegrate. There was a strong possibility that steps to improve disintegration time would affect the self-assembly of the liquid crystalline particles, the stability of those particles and/or the prolonged release of the active ingredient. The ODTs of the present invention has also been designed to maintain the ability to form the self-assembled particles or bulk phase and the prolonged release of the active ingredient.

In some embodiments, the ODT according to the current invention is prepared by spray drying, thermoplastic granulation, wet granulation or any of these processes followed by mixing with further ingredients. Preferred formulations are prepared by wet granulation.

In some embodiments, the ODT of the invention further comprises one or more excipients that do not alter self-assembly of the amphiphilic compound. Optionally, the one or more excipients also do not alter the lattice or water channel size of the liquid crystalline structure formed by the amphiphilic compound. Optionally, the excipients are one or more of Pharmaburst, crospovidone, sodium starch glycolate, sorbitol, silicon dioxide, magnesium stearate, sodium chloride and poly vinyl pyrrolidone.

### Embodiments of Compositions, Self-Assembled Structures and ODTs of the Invention

In all embodiments, the amphiphilic compound is capable of self-assembling in to liquid crystalline particles upon contact with a hydrophilic solvent. “Capable of self-assembly into liquid crystalline particles” refers to being capable of assembling into liquid crystalline particles in hydrophilic solvent of physiological pH, physiological temperature and/or physiological salinity. Optionally, (i) the amphiphile forms liquid crystalline particles at physiological pH, temperature and/or salinity; and/or (ii) the hydrophilic solvent has physiological pH, temperature and/or salinity. It is preferred that, as the composition or tablet (ie of the first, second and fourth aspects of the invention and their embodiments) disintegrates in a hydrophilic solvent the amphiphilic compound self-assembles into liquid crystalline particles. These self-assembled structures optionally are of the third aspect of the invention. It is preferred that the active ingredient is encapsulated into the liquid crystalline particles in the self-assembled structure. The analytical techniques presently available make it difficult to confirm whether or not the active ingredient is encapsulated within the liquid crystalline particles. It is preferred that the composition or tablet is muccoadhesive, that the liquid crystalline particles are muccoadhesive or both. If the tablet is muccoadhesive, it can adhere to human mucosa, which can be observed when the tablet is administered.

In some embodiments, the composition, self-assembled structure or tablet of the invention is for administration to the oral mucosa. Where a composition or tablet of the invention is administered to the oral mucosa, the liquid crystalline particles self-assemble in situ in the oral cavity. One suitable form of administration is sublingual administration (under the tongue). Another suitable form of administration is buccal administration (ie to the buccal vestibule, that is, the area inside the mouth between the lining of the cheek and the teeth/gums). A further form of administration is where the composition is administered under the lip. When administered to the oral mucosa in this way, the active ingredient diffuses into the blood through the mucosa in the mouth. Optionally, the active ingredient is first captured in or attached to a liquid crystalline particle and both the particle and the active diffuses into the mucosa. The active ingredient can enter the blood stream either with or without the liquid crystalline particle also entering the bloodstream.

In some embodiments of the composition, ODT or self-assembled structure of the invention, the self-assembled structure is a cubic phase or hexagonal phase as discussed below. Optionally the self-assembled structure is lamellar phase. Optionally, the self-assembled structure is lamellar phase, a cubic phase or hexagonal phase.

In some embodiments, the composition, ODT or self-assembled structure of the invention is prolonged release. The prolonged release is determined by reference to either an immediate release ODT for administration via the oral mucosa or an immediate release tablet for swallowing.

The amphiphilic compound is a compound that possesses both a hydrophilic portion and a hydrophobic portion and is capable of self-assembling into liquid crystalline particles. The amphiphilic compound can also be a mixture of amphiphiles. Amphiphiles capable of self-assembly behaviour have been described in various publications, such as, for example, Drummond (1999). Examples of amphiphiles that are capable of self-assembly include, but are not limited to: surfactants, lipids, and block copolymers. More specifically, the amphiphilic compound is optionally selected from: fatty acids, fatty alcohols, acylglycerols, glycolipids, sphingolipids, phospholipids, cholesterol and mixtures thereof.

Optionally, the amphiphilic compound is non-ionic.

Hydrophilic-lipophilic balance (HLB) is a measure of the hydrophilicity/lipophilicity of an amphiphile. A HLB under 10 indicates lipid solubility and a HLB over 10 indicates water solubility. Optionally, the amphiphilic compound has a HLB of less than about 10, less than 8 or less than 6. Optionally, the HLB is greater than about 1. Optionally, the HLB is 0 to <10, or 1 to <10, 0 to <8, 1 to <8, 0 to <6 or 1 to <6.

The critical packing parameter (CPP) measures the relative volume of the head (hydrophilic portion) and tail (lipophilic portion) of a surfactant. The CPP indicates the type of liquid crystal likely to form when an amphiphilic compound is in solution at a level above its critical micelle concentration. A CPP of 1 means the surfactant is symmetrical. The CPP is the tail volume (V) divided by the sum of the effective head area (a) and the tail length (I) (ie CPP=V/(a·I)). An amphiphile with a CPP<⅓ is likely to form spherical micelles. An amphiphile with a CPP>⅓ but <½ is likely to form cylindrical micelles. An amphiphile with a CPP>½ but <1 is likely to form lamella micelles. An amphiphile with a CPP>1 is likely to form inversed spherical micelles. The amphiphilic compound optionally has a CPP>⅓, >½, or >1 at body temperature, atmospheric pressure and in water, PBS or saliva. The amphiphilic compound optionally has a CPP>⅓, >½, or >1 at body temperature, atmospheric pressure and in water, PBS, citric acid buffer or saliva.

Optionally, the amphiphilic compound is a non-ionic amphiphile comprising a HLB of 0 to >10 and a CPP of >½. Optionally, the amphiphilic compound is a non-ionic amphiphile comprising a HLB of 1 to >8 and a CPP of >1.

In some embodiments the amphiphilic compound comprises Formula (I):

X−T (I)

wherein

X has at least 2 hydrogen bond forming functional groups and is selected from the group consisting of an ester, ether, anhydride, amide, amine, carbamide, glycerol, biuret, phenyl, pyridine or phosphate; and

T is selected form the group consisting of:

(i) a single C12 to C18 alkyl, alkenyl and alkynyl terminally attached to X optionally comprising:

a. one or more double bonds (preferably cis and at about C7 to C11); or

b. three or more methyl branches (preferably isoprenoid branching);

and

(ii) two C12 to C18 alkyl, alkenyl and alkynyl both terminally attached to X.

The ester and amide groups etc of X can be present in either orientation ie the ester could be —OC(O)-T or —C(O)O-T.

Optionally, the ester, ether, anhydride, amide, amine, carbamide, glycerol, biuret, phenyl, pyridine or phosphate forms part of, or is substituted with, a sugar (eg glucoside), xyloside (monomer or dimer) or C1 to C4 alkyl, alkenyl or alkynyl optionally with two to six hydroxyl, amine or methanol groups and attached at either a terminal or non-terminal carbon.

Optionally, X is selected from the group consisting of ester, ether, amine, amide or glycerol.

Optionally, X has 3 to 6 hydrogen bond forming groups.

Optionally, T has a molecular weight of at least >200 amu.

The amphiphilic compound is optionally selected from the group consisting of glycerol monooleate, glyceryl monolinoleate, glyceryl monooleyl ether, oleyl glycerate, monovaccenin, oleyl urea, linoleyl urea, phytanyl urea, hexahydrofarnesyl-urea, monooleain, phytantriol, glucose stearate, fructose stearate and combinations thereof.

In one embodiment, the amphiphilic compound is selected from a fatty acid comprising a 6 to 24 carbon chain, preferably a 12 to 24 carbon chain, more preferably a 16 to 20 carbon chain, most preferably an 18 carbon chain. The amphiphilic compound can also be a mixture of fatty acids. In a preferred embodiment, the amphiphilic compound is selected from one or more mono- and/or di-glycerides of fatty acids comprising a 6 to 24 carbon chain, preferably a 12 to 24 carbon chain, more preferably a 16 to 20 carbon chain, most preferably an 18 carbon chain. The carbon chain optionally has one or more double bonds such that it is unsaturated. One preferred class of amphiphilic compounds is glycerol monooleates (GMOs). The Handbook of Pharmaceutical Excipients lists GMO as having a HLB of 3.3. In a particularly preferred embodiment the amphiphilic compound is Myverol™ 18-99 k (trade mark owned by Kerry Group Services Limited). Myverol™ is generally considered a GMO despite including some non-GMO amphiphiles. Myverol™ 18-99 k is produced from the reaction of glycerol with canola (low erucic acid rapeseed) oil and contains a mixture of monoacylglycerols, diacylglycerols and glycerol. The compositional analysis of Myverol™ 18-99 k is detailed in Clogston (Clogston 2000) wherein Myverol™ 18-99 k was found to contain 82% monoacylglycerols (consisting of 86.6% monoolein (1-Oleoyl-rac-glycerol), 7.0% monostearin (1-Stearoyl-rac-glycerol), 3.5% monopalmitin (1-monohexadecanoyl-rac-glycerol), 0.9% monoarachidin (1-Arachidonoyl-glycerol) and 2.0% unidentified monoacylglycerols), 13.4% diacylglycerols (consisting of 7.4% 1,2-diacylglycerol and 6.0% 1,3-diacylglycerol) and 4.3% glycerol.

Another grade of GMO suitable for use in the present invention is comprised of about 90-100% monoglycerides (preferably about 95%), about 0-10% diglycerides (preferably about 4%) and about 0-2% triglycerides (preferably about 0.5%). It is preferred if this GMO has not less than 60% methyl oleate (preferably about 65%) and more preferred that the GMO also has not more than 35% methyl linoleate (preferably about 18-20%). The remaining fatty acid composition of the GMO is optionally not more than 12% methyl palmitate (preferably about 4%), not more than 6% methyl stearate (preferably about 2%), not more than 2% methyl linolenate, not more than 2% methyl arachidate, not more than 2% methyl eicosenate and not more than 6% free glycerine (preferably less than 1%).

Thus, in one embodiment the amphiphilic compound is a mixture of amphiphiles. Preferably, the amphiphilic compound contains a mixture of monoacylglycerols, diacylglycerols and glycerol. In particular, the mixture of amphiphiles is produced by reacting glycerol with canola oil. One suitable available amphiphilic compound contains 82% monoacylglycerols, 13.4% diacylglycerols and 4.3% glycerol. More particularly, the amphiphilic compound can contain:

82% monoacylglycerols consisting of 86.6% monoolein, 7.0% monostearin, 3.5% monopalmitin, 0.9% monoarachidin and 2.0% unidentified monoacylglycerols;

13.4% diacylglycerols consisting of 7.4% 1,2-diacylglycerol and 6.0% 1,3-diacylglycerol; and

4.3% glycerol.

In a further embodiment, the amphiphilic compound includes (i) a mixture of a mono- and/or di-glyceride of one or more fatty acids and (ii) one or more free fatty acids. Thus, the amphiphilic compound can include Myverol™ 18-99 k and a fatty acid, such as oleic acid. In a further embodiment, the amphiphilic compound includes monoacylglycerol and oil.

The self-assembled particles are optionally selected from the following group: cubosomes, hexosomes, sponge particles and mixtures thereof, preferably cubosomes.

The self-assembled particles optionally form a bulk phase selected from the group consisting of micellar (normal and reversed), lamellar, hexagonal (normal and reversed), cubic (normal discrete, reversed discrete, reversed bicontinuous—including primitive, gyroid and diamond—and reversed discontinuous), and other ‘intermediate phases’ such as the ribbon, mesh, or non-cubic ‘sponge’ bicontinuous phases. See Israelachvili, J (1994), Chang (1998) and Kaasgard (2006) for more detail. In a preferred embodiment, the bulk phase is selected from cubic phase, hexagonal phase and mixtures thereof, preferably reversed bicontinuous cubic phase, preferably the diamond phase. Optionally, the bulk phase is lamellar phase. Optionally, the bulk phase is lamellar, reversed cubic or reversed hexagonal.

Without being bound by theory or mode of action, it is believed that the more complex the self-assembled particles and/or bulk phase, the slower the release of the active ingredient. Thus, the hexagonal and cubic, particularly diamond cubic, bulk phases are believed to result in the slowest release.

In one embodiment, the active ingredient is incorporated or dissolved within the self-assembled structure. Preferably, the active ingredient is non-covalently incorporated. The active ingredient is optionally in the form of a prodrug. In this embodiment, the active ingredient needs to be cleaved or metabolised, for example by an enzyme or hydrolysis, either before or after absorption to form the active ingredient.

In some embodiments, w/w ratio of amphiphilic compound to active ingredient in the composition, ODT or self-assembled structure of the invention is about 1:1 or more, about 4:1 or more, or about 7:1 or more, about 10:1 or more, about 50:1 or more or about 100:1 or more. Alternatively, the w/w ratio of amphiphilic compound to active ingredient is about 1:1, about 4:1 or about 7:1. It is also possible for the ratio of amphiphilic compound to active ingredient to be lower than 1:1, for example, 1:1.5, 1:2 or 1:3, particularly where the active ingredient has good water solubility. Higher ratios of amphiphilic compound to active ingredient are more likely where the dose of active ingredient is low.

All embodiments, the composition, ODT or self-assembled structure of the invention optionally further comprises additional pharmaceutically acceptable excipients such as one or more filler, binder, glidant, lubricant, osmotic agent, sweetener and/or flavour.

The composition, ODT or self-assembled structure optionally further include a second active ingredient. It is preferred for the active ingredient and optional second active ingredient be micronized. The particle size of the active ingredient and optional second active ingredient in the ODT is preferred to be about 10 μm.

In some embodiments of the composition, ODT or self-assembled structure, the active ingredient is hydrophilic. In alternate embodiments, the active ingredient is lipophilic. For the second to fourth aspects of the invention a hydrophilic active ingredient is preferred.

In some embodiments of the composition, ODT or self-assembled structure of the invention, the active ingredient has a log P of −14 to 6.4, −14 to 0, −0.5 to 6.4, or 0.3 to 6.4. Optionally, in the second to fourth aspects of the invention the active ingredient has a molecular weight of 100 to 10,000 g/mol.

In some embodiments of the composition, ODT or self-assembled structure of the invention, the active ingredient is about 0.05% to about 10% w/w or about 0.1% to about 6% w/w of the ODT. Optionally, the ratio of GMO to active ingredient is about 1:1 to 4:1 by weight.

Suitable active ingredients for the composition, ODT or self-assembled structure of the invention with a molecular weight of 1 to 10 kDa include low molecular weight heparins such as ardeparin, bemiparin, nadroparin, reviparin, enoxaparin, parnaparin, certoprain, dalteparin and tinzaparin. Heparin-related molecules including fondaparinux, heparan sulfate and fractions of heparan sulfate are also suitable.

Suitable active ingredients for other compositions, ODTs or self-assembled structures of the invention include rosuvastatin, fluvastatin, pravastatin, atorvastatin, niacin, amoxicillin, clavulanic acid, trimethoprim, sulfamethoxazole, 5HT2c anti-serotonins, phenteramine, beta blockers, thiazide diuretics, steroids, ACE inhibitors, aspirin, paracetamol and ibuprofen or their derivatives, oxycodone, adrenaline ie epinephrine, melatonin, atenolol, irinotecan, paclitaxel, atropine, haloperidol, levofloxacin, indomethacin, diazepam, trans retinol, prednisolone, progesterone, hydrocortisone, dexamethasone, paracetamol/acetaminophen and capecitabine.

Alternatively, suitable active ingredients for other compositions, ODTs or self-assembled structures of the invention include rosuvastatin, atorvastatin, niacin, oxycodone, adrenaline ie epinephrine, melatonin, atenolol, irinotecan, paclitaxel, atropine, haloperidol, levofloxacin, indomethacin, diazepam, retinol (eg trans retinol), prednisolone, progesterone, hydrocortisone, dexamethasone and/or low molecular weight heparins such as ardeparin, bemiparin, nadroparin, reviparin, enoxaparin, parnaparin, certoprain, dalteparin and tinzaparin.

Preferably, the active ingredient is rosuvastatin, atorvastatin, niacin, oxycodone, enoxaparin and/or dalteparin.

The composition, ODT or self-assembled structure of the invention is optionally mucoadhesive. Further, the composition, ODT or self-assembled structure of the invention is optionally for sublingual administration.

In the composition, ODT or self-assembled structure of the invention, the liquid crystalline particles formed upon contact with a hydrophilic solvent form a bulk phase of liquid crystalline particles. This bulk phase can be cubic. The cubic phase can be Pn3m and/or Im3m.

In the composition, ODT or self-assembled structure of the invention, it is preferred that the amphiphilic compound self-assembles into liquid crystalline particles when contacted with a hydrophilic solvent having physiological pH, physiological temperature and physiological salinity.

The present invention has a number of specific forms. Additional embodiments of these forms are as discussed elsewhere in the specification. The following aspects of the invention further describe options for administration.

An oral disintegrating tablet (ODT) suitable for administration to oral mucosa comprising an amphiphilic compound capable of self-assembling into liquid crystalline particles when contacted with a hydrophilic solvent, a therapeutically effective amount of an active ingredient, a water channel enhancer and a pharmaceutically acceptable disintegrant, wherein the amphiphilic compound is non-ionic. Optionally, the hydrophilic solvent is PBS, citric acid buffer, water and/or saliva.

An oral disintegrating tablet (ODT) suitable for administration to oral mucosa comprising an amphiphilic compound capable of self-assembling into liquid crystalline particles when contacted with a hydrophilic solvent, a therapeutically effective amount of an active ingredient, a water channel enhancer and a pharmaceutically acceptable disintegrant, wherein the amphiphilic compound is GMO, the active ingredient has a log P of −0.5 to 6.4 and the ODT has a hardness of 0.5 to 6 kb. Optionally, the hydrophilic solvent is PBS, citric acid buffer, water and/or saliva.

An oral disintegrating tablet (ODT) suitable for administration to oral mucosa comprising an amphiphilic compound capable of self-assembling into liquid crystalline particles when contacted with a hydrophilic solvent, a therapeutically effective amount of an active ingredient, a water channel enhancer and a pharmaceutically acceptable disintegrant, wherein the amphiphilic compound is GMO, the active ingredient has a log P of −0.5 to 6.4, the ODT has a hardness of 0.5 to 6 kb and the ODT is stable for 3 years. Optionally, the hydrophilic solvent is PBS, citric acid buffer, water and/or saliva.

An oral disintegrating tablet (ODT) for administration to oral mucosa comprising an amphiphilic compound capable of self-assembling into liquid crystalline particles when contacted with a hydrophilic solvent, a therapeutically effective amount of a 1 to 10 kDa active ingredient and a pharmaceutically acceptable disintegrant, wherein the 1 to 10 kDa active ingredient is bioavailable, the amphiphilic compound is GMO, the active ingredient has a log P of −0.5 to 6.4 and the ODT has a hardness of 0.5 to 6 kb. Optionally, the hydrophilic solvent is PBS, citric acid buffer, water and/or saliva.

An oral disintegrating tablet (ODT) for administration to oral mucosa comprising an amphiphilic compound capable of self-assembling into liquid crystalline particles when contacted with a hydrophilic solvent, a therapeutically effective amount of a 1 to 10 kDa active ingredient and a pharmaceutically acceptable disintegrant, wherein the 1 to 10 kDa active ingredient is bioavailable, the amphiphilic compound is GMO, the active ingredient has a log P of −0.5 to 6.4, the ODT has a hardness of 0.5 to 6 kb and the ODT is stable for 3 years. Optionally, the hydrophilic solvent is PBS, citric acid buffer, water and/or saliva.

An oral disintegrating tablet (ODT) suitable for administration to oral mucosa comprising an amphiphilic compound capable of self-assembling into liquid crystalline particles when contacted with a hydrophilic solvent, a therapeutically effective amount of an active ingredient, a water channel enhancer and a pharmaceutically acceptable disintegrant, wherein the amphiphilic compound is selected from the group consisting of glycerol monooleate, glyceryl monolinoleate, glyceryl monooleyl ether, oleyl glycerate, monovaccenin, oleyl urea, linoleyl urea, phytanyl urea, hexahydrofarnesyl-urea, monooleain, phytantriol, glucose stearate, fructose stearate and combinations thereof, the active ingredient has a log P of −0.5 to 6.4 and the ODT has a hardness of 0.5 to 6 kb. Optionally, the hydrophilic solvent is PBS, citric acid buffer, water and/or saliva.

An oral disintegrating tablet (ODT) for administration to oral mucosa comprising an amphiphilic compound capable of self-assembling into liquid crystalline particles when contacted with a hydrophilic solvent, a therapeutically effective amount of a 1 to 10 kDa active ingredient and a pharmaceutically acceptable disintegrant, wherein the 1 to 10 kDa active ingredient is bioavailable, the amphiphilic compound is selected from the group consisting of glycerol monooleate, glyceryl monolinoleate, glyceryl monooleyl ether, oleyl glycerate, monovaccenin, oleyl urea, linoleyl urea, phytanyl urea, hexahydrofarnesyl-urea, monooleain, phytantriol, glucose stearate, fructose stearate and combinations thereof, the active ingredient has a log P of −0.5 to 6.4 and the ODT has a hardness of 0.5 to 6 kb. Optionally, the hydrophilic solvent is PBS, citric acid buffer, water and/or saliva.

An oral disintegrating tablet (ODT) suitable for administration to oral mucosa comprising an amphiphilic compound capable of self-assembling into liquid crystalline particles when contacted with a hydrophilic solvent, a therapeutically effective amount of an active ingredient, a water channel enhancer and a pharmaceutically acceptable disintegrant, wherein the amphiphilic compound is non-ionic, the ODT disintegrates in less than 2 minutes following contact with a hydrophilic solvent and the ODT prolongs the release of the active ingredient. Optionally, the hydrophilic solvent is PBS, citric acid buffer, water and/or saliva.

An oral disintegrating tablet (ODT) suitable for administration to oral mucosa comprising an amphiphilic compound capable of self-assembling into liquid crystalline particles when contacted with a hydrophilic solvent, a therapeutically effective amount of an active ingredient, a water channel enhancer and a pharmaceutically acceptable disintegrant, wherein the amphiphilic compound is GMO, the active ingredient has a log P of −0.5 to 6.4, the ODT has a hardness of 0.5 to 6 kb, the ODT disintegrates in less than 2 minutes following contact with a hydrophilic solvent and the ODT prolongs the release of the active ingredient. Optionally, the hydrophilic solvent is PBS, citric acid buffer, water and/or saliva.

An oral disintegrating tablet (ODT) suitable for administration to oral mucosa comprising an amphiphilic compound capable of self-assembling into liquid crystalline particles when contacted with a hydrophilic solvent, a therapeutically effective amount of an active ingredient, a water channel enhancer and a pharmaceutically acceptable disintegrant, wherein the amphiphilic compound is GMO, the active ingredient has a log P of −0.5 to 6.4, the ODT has a hardness of 0.5 to 6 kb, the ODT disintegrates in less than 2 minutes following contact with a hydrophilic solvent and the ODT prolongs the release of the active ingredient and the ODT is stable for 3 years. Optionally, the hydrophilic solvent is PBS, citric acid buffer, water and/or saliva.

An oral disintegrating tablet (ODT) for administration to oral mucosa comprising an amphiphilic compound capable of self-assembling into liquid crystalline particles when contacted with a hydrophilic solvent, a therapeutically effective amount of a 1 to 10 kDa active ingredient and a pharmaceutically acceptable disintegrant, wherein the 1 to 10 kDa active ingredient is bioavailable, the amphiphilic compound is GMO, the active ingredient has a log P of −0.5 to 6.4, the ODT has a hardness of 0.5 to 6 kb, the ODT disintegrates in less than 2 minutes following contact with a hydrophilic solvent and the ODT prolongs the release of the active ingredient. Optionally, the hydrophilic solvent is PBS, citric acid buffer, water and/or saliva.

An oral disintegrating tablet (ODT) for administration to oral mucosa comprising an amphiphilic compound capable of self-assembling into liquid crystalline particles when contacted with a hydrophilic solvent, a therapeutically effective amount of a 1 to 10 kDa active ingredient and a pharmaceutically acceptable disintegrant, wherein the 1 to 10 kDa active ingredient is bioavailable, the amphiphilic compound is GMO, the active ingredient has a log P of −0.5 to 6.4, the ODT has a hardness of 0.5 to 6 kb, the ODT is stable for 3 years, the ODT disintegrates in less than 2 minutes following contact with a hydrophilic solvent and the ODT prolongs the release of the active ingredient. Optionally, the hydrophilic solvent is PBS, citric acid buffer, water and/or saliva.

An oral disintegrating tablet (ODT) suitable for administration to oral mucosa comprising an amphiphilic compound capable of self-assembling into liquid crystalline particles when contacted with a hydrophilic solvent, a therapeutically effective amount of an active ingredient, a water channel enhancer and a pharmaceutically acceptable disintegrant, wherein the amphiphilic compound is selected from the group consisting of glycerol monooleate, glyceryl monolinoleate, glyceryl monooleyl ether, oleyl glycerate, monovaccenin, oleyl urea, linoleyl urea, phytanyl urea, hexahydrofarnesyl-urea, monooleain, phytantriol, glucose stearate, fructose stearate and combinations thereof, the active ingredient has a log P of −0.5 to 6.4, the ODT has a hardness of 0.5 to 6 kb, the ODT disintegrates in less than 2 minutes following contact with a hydrophilic solvent and the ODT prolongs the release of the active ingredient. Optionally, the hydrophilic solvent is PBS, citric acid buffer, water and/or saliva.

An oral disintegrating tablet (ODT) for administration to oral mucosa comprising an amphiphilic compound capable of self-assembling into liquid crystalline particles when contacted with a hydrophilic solvent, a therapeutically effective amount of a 1 to 10 kDa active ingredient and a pharmaceutically acceptable disintegrant, wherein the 1 to 10 kDa active ingredient is bioavailable, the amphiphilic compound is selected from the group consisting of glycerol monooleate, glyceryl monolinoleate, glyceryl monooleyl ether, oleyl glycerate, monovaccenin, oleyl urea, linoleyl urea, phytanyl urea, hexahydrofarnesyl-urea, monooleain, phytantriol, glucose stearate, fructose stearate and combinations thereof, the active ingredient has a log P of −0.5 to 6.4, the ODT has a hardness of 0.5 to 6 kb, the ODT disintegrates in less than 2 minutes following contact with a hydrophilic solvent and the ODT prolongs the release of the active ingredient. Optionally, the hydrophilic solvent is PBS, citric acid buffer, water and/or saliva.

Methods & Uses of the Invention

The invention also provides for the use of the composition, ODT or self-assembled structure of the invention as described in any one of the embodiments in this specification in treating or preventing a disease state. Optionally, in a subject in need thereof.

The invention further provides a method of administering an active ingredient via the oral mucosa comprising administration of a composition, ODT or self-assembled structure of the invention. Optionally the active ingredient is delivered systemically via the oral mucosa.

The invention further provides a composition, ODT or self-assembled structure of the invention for administration to the oral mucosa, wherein the active ingredient is delivered systemically via the oral mucosa.

The invention further provides use of an active ingredient in the preparation of a composition, ODT or self-assembled structure of the invention for administration to the oral mucosa, wherein the active ingredient is delivered systemically via the oral mucosa.

The active ingredient administered to the oral mucosa in these methods and uses is bioavailable. Bioavailability for this transmucosal dosage form can be different to oral bioavailability for other oral dosage forms. The presence of the self-assembled structures enables the delivery of active ingredients that are not orally bioavailable in other environments. It will be evident to the skilled person that oral bioavailability requires that the active ingredient not be sequestered in the liquid crystalline particles formed when the ODT contacts saliva but that where the active ingredient is encapsulated in the liquid crystalline particles it is released from those particles with suitable speed. Bioavailability can be tested in vitro using the Ussing chamber testing set out in the examples.

In an alternate aspect, the invention provides a method of administering an active ingredient to a mammal, particularly a human, comprising administration of the composition, ODT or self-assembled structure of the invention to the oral mucosa of the mammal. Other embodiments of the composition, ODT or self-assembled structure of the invention used in the method are as described throughout this patent. Optionally, prior to the administration the mammal is selected as being in need of the treatment.

Where the active ingredient is a statin (preferably rosuvastatin), the method or use is preferred to reduce a subject's total cholesterol, treat or prevent the development of dyslipidaemia, preferably hyperlipidaemia, cardiovascular disease and/or atherosclerosis. Alternatively or in addition, the method can treat or prevent statin-intolerance, preferably statin-induced myalgia, statin-induced myositis and/or statin-induced myopathy. Further support for these methods is in international patent application no PCT/AU2018/050367.

Where the active ingredient is oxycodone, the method or use is preferred to be for the treatment of pain or for analgesic effect.

Where the active ingredient is a low molecular weight heparain, for example, enoxaparin and/or dalteparin, the method or use is preferred to be for one or more of the prevention of blood clots, treatment of venous thromboembolism and treatment of myocardial infarction.

Where the active ingredient is adrenalin or epinephrine, the method or use is preferred to be for one or more of the prevention and/or treatment of anaphylaxis, cardiac arrest and superficial bleeding.

In a further aspect, the present invention provides a method for confirming that an ODT of the invention self assembles into liquid crystalline particles following contact with a hydrophilic solvent comprising dissolving an ODT according to the invention in a hydrophilic solvent (eg saliva or PBS) to produce a suspension and analysing the suspension using the SAXS/WAXS beamline of a synchrotron to determine if liquid crystalline particles are present. Optionally, the exposure time is 5 seconds. Optionally the suspension is prepared at ambient temperature (eg about 22° C.) and the analysis occurs at ambient temperature (eg about 22° C.).

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1—Outline of the lyotropic liquid crystalline phases that can be formed when water is added to an anhydrous lipid. Normal (o/w) phases are designed I and inversed (w/o) phases II with decreasing packing parameter as water concentration increases. Adapted with permission from Israelachivili et al. (1994) and Nguyen (2009). Israelachvili, J., The science and applications of emulsions—an overview. Colloids Surf. Physico chem. Eng. Aspects 1994, 91, 1-8. Nguyen, T.-H. Investigation of novel liquid crystalline materials for the sustained oral delivery of poorly water soluble drugs. PhD, Monash University, Melbourne, 2009.

FIG. 2—Diagrammatic representation of the structure of the three main bicontinuous and two main hexagonal crystal structures where (a) Gyroid (G) Ia3d, (b) Diamond (D) Pn3m and (c) Primitive (P) Im3m. The hexagonal liquid crystals are represented by (d) inverse and (e) normal hexagonal structure. Diagram adapted from Caffrey and Cheng (1995) and Nguyen (2009).

FIG. 3—Appearance of rosuvastatin in the receptor chamber after application of mixtures containing GMO and rosuvastatin at a ratio of 1:1 (closed circle), 5:1 (open circle) and 7:1 (closed triangle) to porcine buccal mucosa in the donor chamber of a modified Ussing Chamber. Data are presented as mean±SD (n=4-5).

FIG. 4—The appearance of rosuvastatin in the receptor chamber after application of SBT tablets 122 (closed circle), 123 (open circle), 137 (closed triangle), 138 (open triangle) to porcine buccal mucosa in the donor chamber of a modified Franz Cell. Data are presented as mean±SEM (n=3-5). The closed square, open square and closed diamond and not relevant.

FIG. 5—Graph of the highest average peak area from the HPLC on samples from the receptor chamber in the experiment graphed in FIG. 3. From the left: GMO:API 1:1 mix; GMO:API 5:1 mix; GMO:API 7:1 mix.

FIG. 6—Graph of the highest average peak area from the HPLC on samples from the receptor chamber in the experiment graphed in FIG. 4. From the left: GMO:API 1:1 mix; GMO:API 7:1 mix; SBT122; SBT123.

FIG. 7—The appearance of oxycodone in the receptor chamber over time after application of SBT227 (diamond) and SBT232 (square) to porcine buccal mucosa in the donor chamber of a Ussing chamber. Data are presented as mean±SEM (n=5). Both formulations demonstrate slow release characteristics. SBT227 release the API relatively faster comparing to SBT232 due to lower quantity of GMO.

FIG. 8—The appearance of atorvastatin in the receptor chamber over time after application of SBT226 (square) and SBT233 (diamond) to porcine buccal mucosa in the donor chamber of a Ussing chamber. Data are presented as mean±SEM (n=5). Both formulations demonstrate slow release characteristics. SBT226 release the API relatively faster comparing to SBT233 due to lower quantity of GMO.

FIG. 9—Plot of the percentage of enoxaparin in receptor chamber following application of Formula 1 to the mucosa. Results are based upon the total enoxaparin sodium added to the donor chamber at the start of the experiment. These results are tabulated in Table 31. The top (ie highest percentage release) results are from cell 3, the middle results from cell 1 and the lowest results from cell 2).

FIG. 10—Plot of the percentage of enoxaparin in receptor chamber following application of Formula 2 to the mucosa. Results are based upon the total enoxaparin sodium added to the donor chamber at the start of the experiment. These results are tabulated in Table 35. The top (ie highest percentage release) results are from cell 1 and the lowest results from cell 2).

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

Further aspects of the present invention and further embodiments of the aspects described in the preceding paragraphs will become apparent from the following description, given by way of example.

All of the patents and publications referred to herein are incorporated by reference in their entirety.

For purposes of interpreting this specification, terms used in the singular will also include the plural and vice versa.

One skilled in the art will recognize many methods and materials similar or equivalent to those described herein, which could be used in the practice of the present invention. The present invention is in no way limited to the methods and materials described.

The inventors of the present invention have developed an ODT comprising a large API for the delivery of active ingredients via the oral mucosa of a subject. The ODT disintegrates and forms liquid crystalline particles when it contacts a hydrophilic solvent. The liquid crystalline particles that form when the ODT contacts a hydrophilic solvent optionally also have increased water channel size. The ODT comprising an amphiphilic compound capable of self-assembling into liquid crystalline particles when contacted with a hydrophilic solvent, a therapeutically effective amount of an active ingredient, a water channel enhancer, and a pharmaceutically acceptable disintegrant that results in the formation of liquid crystalline particles with increased lattice size/water channel size are optionally be prepared with either large or small molecule active ingredients.

The term “therapeutically effective amount” as used throughout the specification is understood to mean the amount or dose of a compound or composition thereof that will lead to one or more desired effects, for example, the reduction of cholesterol synthesis. A therapeutically effective amount of an active ingredient will vary according to factors such as the disease state, age, sex, and weight of a subject, and the ability of the substance to elicit a desired response in the subject.

The term “dalteparin” refers to a low molecular weight heparin having a molecular weight of 5.6-6.4 kDa. An example is marketed as Fragmin by Pfizer Inc.

The term “water channels” refers to the channels of hydrophilic solvent through a complex liquid crystalline particle or bulk phase, such as a cubic or hexagonal particle/phase, assembled in hydrophilic solvent.

The term “water channel enhancer” refers to an ingredient that increases the size of the repetitive lattice structure and/or water channel size within a complex liquid crystalline particle or bulk phase, such as a cubic or hexagonal particle/phase. The size of the usual water channel/lattice for a specific amphiphile in certain conditions (eg temperature) can be determined by combining that amphiphile with a hydrophilic solvent so that it self-assembles into liquid crystalline particles and then analysing the particles on the SAX/WAX beamline of a synchrotron and analysing the results as described below and in the examples. A compound can be tested for water channel enhancing effect by combining the compound with the amphiphile and the hydrophilic solvent and then testing on the SAX/WAX beamline of a synchrotron and analysing the results as described below and in the examples to determine if the water channel and/or lattice size has increased. Many water channel enhancers have a threshold effective concentration. This concentration can also be determined experimentally. When reference is made to an increase in the water channel and/or lattice size for the liquid crystalline structure formed by an amphiphile, this refers to an increase in comparison to the size of the water channel and/or lattice size for the same amphiphile in the same conditions except without the presence of the water channel enhancer.

As used herein, the terms “amphiphilic compound” and “amphiphile” are used interchangeably.

The term “enoxaparin” and “enoxaparin sodium” refer to a low molecular weight heparin and its sodium salt having a molecular weight of 3.8-5.0 kDa. Examples are marketed as Clexane and Xaparin.

The term “low molecular weight heparin” refers to fractionated heparin where at least 60% of all chains have a molecular weight less than 8 kDa. Optionally, at least 70%, at least 80% or at least 90% of all chains have a molecular weight less than 8 kDa. Examples include ardeparin, bemiparin, nadroparin, reviparin, enoxaparin, parnaparin, certoprain, dalteparin and tinzaparin. The low molecular weight heparin can be a heparin-related molecule.

The term “heparin” refers to a linear sulfated glycosaminoglycan polymer made up of the following structure:

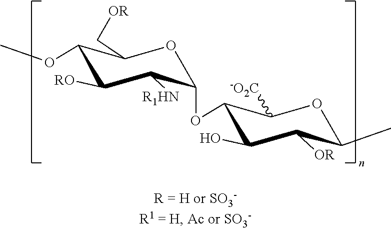

The most common disaccharide unit of heparin is depicted below:

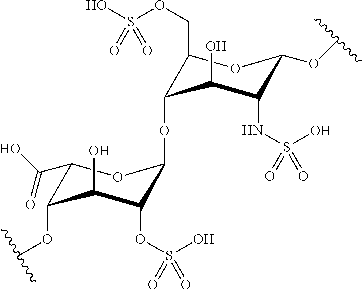

Other common substitution patterns present in the disaccharide units of heparin include GlcA-GlcNAc, GlcA-GlcNS, IdoA-GlcNS, IdoA(2S)-GlcNS, IdoA-GlcNS(6S) and IdoA(2S)-GlcNS(6S); wherein GlcA is β-D-glucuronic acid, IdoA is α-L-iduronic acid, IdoA(2S) is 2-O-sulfo-α-L-iduronic acid, GlcNAc is 2-deoxy-2-acetamido-α-D-glucopyranosyl, GlcNS is 2-deoxy-2-sulfamido-α-D-glucopyranosyl and GlcNS(6S) is 2-deoxy-2-sulfamido-α-D-glucopyranosyl-6-O-sulfate. Heparin also includes disaccharide units containing a 3-O-sulfated glucosamine (GlcNS(3S,6S)) or a free amine group (GlcNH₃⁺) in low concentrations.

Unfractionated heparin is heparin having a molecular weight of 12 to 15 kDa.

The term “heparin-related molecule” refers to an oligomer or polymer substantially related to heparin or a fragment of heparin. Examples include fondaparinux, heparan sulfate and fractions of heparan sulfate.

The term “self-assembled particles” as used throughout the specification is understood to mean an aggregate of amphiphiles that possess some degree of internal organisational order, for example, a liquid crystalline or colloidal particle or colloidosome or a solid lipid particle. The particles can be either nanoparticles or microparticles depending on their average size, typically less than about 1 μm, preferably in a range of about 10 nm to about 500 nm, more commonly about 200 nm. The self-assembled particles are formed by contacting the amphiphile with solvent (ie lyotropic liquid crystalline particles). In some embodiments, the self-assembled particles themselves aggregate into a bulk lyotropic phase.

The term “liquid crystalline particles” as used throughout the specification is understood to mean self-assembled particles including liquid crystalline structure.

The term “bulk phase” as used throughout the specification is understood to mean a lyotropic phase that includes but is not limited to: micellar cubic (I1); normal hexagonal (H1); bicontinuous cubic (V1); lamellar (L); reversed bicontinuous cubic (V2); reversed hexagonal (H2); reversed micellar cubic (I2) and sponge (L3) phases.

The term “cubic phase” as used throughout the specification is understood to refer to two main classes of phases: micellar cubic (11) and bicontinuous cubic (V1). ‘Micellar cubic phase’ refers to a phase consisting of spherical micelles arranged in a cubic array. A ‘normal micellar cubic phase’ or ‘II phase’ (ie (11)) consists of spherical normal micelles arranged in a cubic array, whilst an ‘inverse micellar cubic phase’ or ‘III phase’ (ie (I2)) consists of spherical inverse micelles arranged in a cubic array. ‘Bicontinuous cubic phase’ refers to a family of closely related phases that consist of a single curved lipid bilayer that forms a complex network that separates the polar solvent space into two continuous, but non-intersecting volumes. Bicontinuous cubic phases possess long range order based upon a cubic unit cell. Bicontinuous cubic phases have zero mean curvature; that is, at all points on surface of the amphiphile bilayer, the surface is as convex as it is concave. Bicontinuous cubic phases include the normal (‘vI phase’ or ‘V1 phase’) or reverse (‘vII phase’ or ‘V2 phase’) type. Several types of long range orientational orders have been observed for bicontinuous cubic phases; the orientational order in these phases correspond to space groups Ia3d, Pn3m, and Im3m. When a colloidosome possesses the internal structure of a bulk cubic phase the colloidosome is referred to as a ‘cubosome’.

The term “hexagonal phase” as used throughout the specification is to be understood to mean an amphiphile phase consisting of long, rod-like micelles packed into a hexagonal array. A “normal hexagonal phase” is a hexagonal phase consisting of long, rod-like normal micelles, whilst an ‘inverse hexagonal phase’ is a hexagonal phase consisting of long, rod-like inverse micelles. The normal hexagonal phase is also referred to as the “HI phase” or “H1 phase” and the inverse hexagonal phase is also referred to as the “HII phase” or “H2 phase”. When a colloidosome possesses the internal structure of a bulk hexagonal phase the colloidosome is referred to as a ‘hexosome’.

The term “lamellar phase” or “L phase” as used throughout the specification is to be understood to mean a stacked bilayer arrangement, where opposing monolayers of the hydrophilic portion of amphiphile molecules are separated by a polar solvent domain, while the hydrophobic portion of the amphiphile molecule of the back-to-back layers are in intimate contact to form a hydrophobic layer. The planar lamellar phase is referred to as the “Lα phase”. There are three lamellar phases, (1) the fluid lamellar phase (Lα) where the chains are melted, (2) the gel lamellar phase (Lβ) where the chains are mostly melted but some degree of short range order and (3) the lamellar crystalline phase (Lc), where the chains are crystalline with very short range order.

The term “sponge phase” or “L3 phase” as used throughout the specification refers to a phase that resembles a bicontinuous cubic phase, in that it possesses an amphiphile bilayer that separates the polar solvent space into two unconnected volumes, but it does not possess long range order. Accordingly, these phases are analogous to a ‘melted cubic phase’.

The term “prodrug” as used throughout the specification refers to a biologically active agent including structural modifications thereto, such that in vivo the prodrug is converted, for example, by hydrolytic, oxidative, reductive or enzymatic cleavage to the biologically active agent by one or more reactions or steps. It includes an agent that requires one or more chemical conversion steps or steps of metabolism to produce the active molecule.

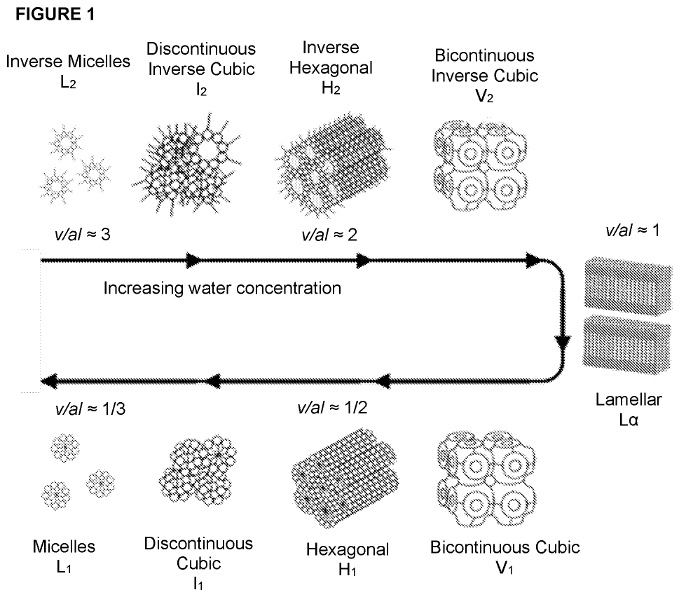

The term “disintegrant” as used throughout the specification refers to a tabletting excipient that is incorporated into the formulation of tablets or capsules to promote their disintegration when they come into contact with liquid or fluid matter, specifically aqueous liquid. The general purpose of incorporating one or more disintegrants in the product formulation is to increase the surface area of the product and soften the binding matter that holds together the solid particles that make up the product. The rate of dissolution in the media increases as the particle size reduces and is greatest when the tablets or capsules reduced to fine particles, as shown schematically in Figure. 1. Rapid dissolution increases the rate of absorption of the active ingredient by the body, producing the desired therapeutic action. Tablets that are labelled as chewable generally do not require a disintegrant to be incorporated in the formulation.

The terms “oral disintegrating tablet”, “orally dispersible tablet”, “fast dissolving tablet”, or “ODT” as used throughout the specification refer to tablets that disintegrate rapidly in the oral cavity of a subject after administration. ODTs do not need to be swallowed. No liquid is required to be consumed by the subject when taking medication from an ODT either. The ODTs of the invention are for delivery of an API via the oral mucosa. However, traditional ODTs were designed to ease swallowing of an API.

The term “substantially disintegrates” as used throughout the specification refers to a disintegration of the tablet largely into constituent particles which were previously compressed into whole tablets.

The term “drug”, “active” or “active pharmaceutical ingredient” as used herein includes a pharmaceutically acceptable and therapeutically effective compound suitable for treating or preventing one or more diseases, symptoms of diseases, and medical conditions, as well as pharmaceutically acceptable salts, stereoisomers and mixtures of stereoisomers, solvates (including hydrates), and/or esters thereof.

The liquid crystalline particles of the present invention are optionally self-assembled into bulk phase including an active ingredient. Typically, a bulk material having a certain phase will form from an amphiphile, that is, a molecule that possesses both a hydrophilic portion and a hydrophobic portion. The self-assembly behaviour of amphiphiles in solvent arises because of the preferential interaction between the solvent and either the hydrophilic or hydrophobic portion of the amphiphilic molecule. When an amphiphile is exposed to a polar solvent, the hydrophilic portion of the amphiphile tends to preferentially interact with the polar solvent, resulting in the formation of hydrophilic domains. The hydrophobic portion of the amphiphile molecules tend to be excluded from this domain, resulting in the de facto formation of a hydrophobic domain.

It is in a self-assembled form (ie as liquid crystalline particles or liquid crystalline bulk phase) that amphiphiles are capable of acting as an inert carrier or matrix into which biologically active molecules, such as an active ingredient, are incorporated. The nanoscale porosity of the self-assembled materials provides a high internal and external surface area. An active ingredient that is distributed within a region of this material is believed to be distributed in an ordered arrangement, and at a high loading concentration due to the large internal and external liquid crystal surface area. Self-assembled bulk phases exist with a variety of orientational orders. If long-range orientational order is observed within the self-assembled bulk phase at equilibrium, the self-assembled bulk phase is termed a ‘mhesophase’, a ‘lyotropic liquid crystalline phase’, a ‘lyotropic phase’ or, as used herein, simply a ‘phase’.

There are 2 principal types of liquid crystalline phases: thermotropic liquid crystals and lyotropic liquid crystals. Thermotropic liquid crystals can be formed by heating a crystalline solid or by cooling an isotropic melt of an appropriate solute. Lyotropic liquid crystals can be formed by addition of a solvent to an appropriate solid or liquid amphiphile. The manipulation of parameters such as amphiphile concentration and chemical structure, solvent composition, temperature and pressure can result in the amphiphile-solvent mixture adopting lyotropic phases with distinctive characteristics.

Examples of particular phases that can be formed by self-assembled particles are set out above. It is possible to disperse the bulk phases described above to form colloidal particles (so-called ‘colloidosomes’) that retain the internal structure of the non-dispersed bulk phase. When these particles possess the internal structure of a reversed bicontinuous cubic phase, the particles are colloquially referred to as cubosomes. Similarly, when the particles possess the internal structure of a reversed hexagonal phase, they are referred to as hexosomes. When the particles possess the internal structure of a lamellar phase, they are referred to as liposomes.

Whilst the bulk materials can be of use in some circumstances, the use of bulk materials having cubic phases in drug administration is limited by their high viscosity making them difficult to administer. In these cases, colloidal dispersions of particles of these phases can optionally be used in drug delivery. More preferred phases for use as drug delivery vehicles when prolonged release of the active ingredient is desired are bicontinuous cubic phase or reversed hexagonal phase. The inverse cubic phase affords distinct aqueous regions that form two continuous water networks (or channels) throughout the cubic phase that more readily allow diffusion of an active ingredient. The inverse cubic liquid crystal phase is thermodynamically stable and co-exists in equilibrium with excess water over a broad temperature range. Alternatively, if the bicontinuous cubic phase is viscous and difficult to administer it could be possible to administer a lamellar phase material that converts into the cubic phase upon dissolution with aqueous, water rich, body fluids (thus facilitating the conversion of one phase to another). For example, a suitable material is a phospholipid such as 1,2-dioleoyl-sn-glycero-3-phosphocholine. The cubic phase in situ provides a viscous depot from which an active ingredient can slowly be released. An inverse cubic liquid crystal phase provides an appropriate scaffold in which to distribute or load the active ingredient owing to the high surface area of the internal liquid crystal structure (up to 400 m²/g).

Suitable pharmaceutical carriers, excipients, diluents, additives and vehicles are known to those skilled in the art.

The formulation optionally includes one or more binders such as hydroxypropylmethylcellulose (HPMC), ethyl cellulose, acacia, polyvinyl alcohol (PVA), and polyvinylpyrrolidone (Povidone). Povidone is a preferred binder. Optionally, the binder is about 0.5 to about 5% w/w of the ODT. Optionally, the binder is about 1 to about 3% w/w or about 1.5 to about 2.5% w/w of the ODT.

The formulation optionally includes one or more glidants such as talc, magnesium trisilicate and colloidal silicon dioxide.

The formulation optionally includes one or more fillers such as lactose, mannitol, sorbitol, starch, maltodextrin, acacia and silicon dioxide.

The formulation optionally includes one or more lubricants such as glyceryl behenate, stearic acid, talc, zinc stearate, calcium stearate, magnesium stearate, aluminum stearate and sodium stearyl fumarate.

If the formulation is prepared by thermoplastic granulation the formulation optionally includes thermoplastic granulation agents such as glycerol monostearate and glyceryl behenate.

The presence of liquid crystalline phase or a colloidal dispersion of liquid crystalline particles in a self-assembled structure of the invention or following contact of a composition or ODT of the invention with a hydrophilic solvent can be determined using the SAX/WAX beamline of a synchrotron, cross polarised light microscopy (CPLM) or Cry-Em. In certain circumstances, such as a low proportion of amphiphilic compound in the ODT, liquid crystalline phase may not be identified using the SAX/WAX beamline of the synchrotron and an alternative, such as, CPLM may be preferred. CPLM can identify LC structures but does not provide information on the internal phase.

Properties of liquid crystals have been extensively characterized using small angle x-ray scattering (SAXS). SAXS is a non-destructive method to evaluate the shape, size, internal structure, crystallinity and porosity of a nanostructured sample. An x-ray beam is fired at the sample and the intensity of the scattered x-rays are recorded according to Bragg's law;

2d sin θ=nλ

Where n is an integer, λ is the wavelength, θ is the scattering and d is the interplanar distance. The scattering pattern is converted to a plot of intensity versus scattering vector, q, by equation:

q=4πλ sin θ/2

The scattering vector where peaks are located can be used to calculate d by equation:

d=2π/q

Through integration, the pattern intensity vs. scattering vector (q) plot is generated. The unique spacing of peaks act as a ‘fingerprint’ to accurately determine the liquid crystalline structure present.

Using Bragg's law, the lattice parameter, a, can be calculated.

While lattice parameter acts as an indication for water channel size, accurate calculations can be performed using the following equations:

(Pn3m)r=0.391α−I

(Im3m)r=0.305α−I

Where, r=the radius of the water channel, a=the lattice parameter (determined via SAXS) and I=lipid chain length. The lipid chain length (I) of GMO is about 18 Angstrom.

The release of an active ingredient depends on both the liquid crystalline structure and the water channel size. For a specific amphiphile, the water channel size varies between the different liquid crystalline structures as follows:

H2<Ia3d<Pn3m<Im3m.

The cubic structures (Ia3d, Pn3m, Im3m) have bicontinuous water channels (ie 2 water channels in a maze-like configuration) whereas H2 has smaller discontinuous water channels. Most of the water channels in H2 liquid crystalline particles are closed. The closed water channels is one of the reasons for the slow release of active ingredient from this structure.

The CPP of an amphiphilic compound can be determined by quantum mechanics molecular simulations to determine geometrical and quantitative structure-activity relationship (QSAR) values. See, Fong 2016. The HLB of an amphiphilic compound is calculated based on the number and identity of hydrophilic/lipophilic groups. The skilled person understand these calculations.

The CPP and HLB of some amphiphilic compounds are in Table A.

TABLE A
CPP and HLB for various amphiphilic compounds

| Amphiphile | CPP | V | A₀ | L_{C} | HLB |
|---|---|---|---|---|---|
| Phytantriol | 0.650 | 303.5 | 27.9 | 16.8 | 6.36 |
| Monolinolein | 1.016 | 341.0 | 22.6 | 14.8 | 1.02 |
| Glucose stearate | 0.456 | 315.3 | 31.2 | 22.2 | 9.28 |
| Fructose stearate | 0.421 | 315.3 | 33.8 | 22.2 | 9.28 |

The active ingredients melatonin and atenolol have been shown to load and release from a monoolein-water liquid crystalline system previously and are expected to be compatible with the ODT of this invention. Atropine, haloperidol, levofloxacin, indomethacin, diazepam, trans retinol, prednisolone, progesterone, hydrocortisone and dexamethasone have been shown to load an release from monoolein and/or phytantriol liquid crystals previously and are expected to be compatible with the ODT of this invention. Irinotecan and paclitaxel has also been released from inverse hexagonal phase previously and are expected to be compatible with the ODT of this invention. Niacin has been shown to load and release from inverse hexagonal phase (see WO 2014/179845). The log P and molecular weight for some active ingredients are in Table B below.

TABLE B
LogP and molecular weight for various active ingredients

| API | MW (g/mole) | logP |
|---|---|---|
| Atorvastatin | 558.6 | 6.36 |
| Oxycodone | 315.4 | 1.03 |
| Adrenaline | 333.3 | −0.5 to −1.4 |
| Heparin | — | −13.2 |
| Rosuvastatin | 521.6 | 1.92 |
| Niacin | 123.1 | 0.36 |

The molecular weight for some large active ingredients are in Table C below.

TABLE C
Molecular weight of large active ingredients

| API | MW (g/mole) | |
|---|---|---|
| Enoxaparin | 3.8-5.0 | kDa |
| Dalteparin | 5.6-6.4 | kDa |
| Low molecular weight | Up to 8 | kDa |
| heparin | | |

Palatability Requirements

Because ODT compositions disintegrate in the mouth of the patient, it is highly preferable for ODT compositions to be palatable, that is, have acceptable organoleptic attributes such as taste, after-taste, “mouthfeel”, disintegration time, and Flavour-Sweetener Balance (FSB). The FSB scale ranges from 4 (low, need to increase), 7 (optimum), and 10 (over-powered, need to reduce).

Taste and after-taste attributes are defined using a bitterness scale of 1 to 10, wherein 1 is extremely bitter; taste-masking required, 5 is acceptable, and 10 is pleasant tasting. The mouthfeel scale ranges from 1 (very gritty, unacceptable), 5 (non-gritty), and 10 (creamy, smooth).

The ODTs of the present invention have taste and aftertaste attributes at least about 5, at least about 6, at least about 7, at least about 8, at least about 9, or about 10.

The ODTs of the present invention have mouthfeel attributes of at least about 5, at least about 6, at least about 7, at least about 8, at least about 9, or about 10. The ODT compositions of the present invention have FSB attributes of about 5 to about 9, or about 6 to about 8, or about 7.

Optionally, the compositions, self-assembled particles and ODTs of the invention do not include non-API proteins and/or tocopherol. Optionally, the compositions, self-assembled particles and ODTs of the invention do not include poloxamers and/or surfactants other than the amphiphilic compound.

Preparation of the ODTs of the Invention

The ODTs of the invention can be prepared in accordance with the methods described in the examples and those in International patent application no PCT/AU2018/050364. The skilled person is able to determine when in the processing the water channel enhancer should be added based on the nature of the ODT (ie active ingredient or sweetener etc) and the properties of the water channel enhancer.

Compositions of the invention can be prepared by blending the amphiphile with the active ingredient and then the water channel enhancer or vice versa or blending all three ingredients at the same time. If the amphiphile is viscous or semi-solid the amphiphile may require heating before blending.

A self-assembled structure of the invention can be prepared by contacting an ODT or composition of the invention with a hydrophilic solvent or by adding an amphiphile to hydrophilic solvent containing the active ingredient and water channel enhancer.

### REFERENCES

The text of each of the following references is incorporated by reference into this specification.
Caffrey, M.; Cheng, A., Kinetics of lipid phase changes. Curr. Opin. Struct. Biol. 1995, 5, 548-555.
Chang, C.-M.; Bodmeier, R., Low viscosity monoglyceride-based drug delivery systems transforming into a highly viscous cubic phase. Int. J. Pharm. 1998, 173, 51-60.
Clogston, J.; Rathman, J.; Tomasko, D.; Walker, H.; Caffrey, M., Phase behavior of a monoacylglycerol (Myverol 18-99K)/water system. Chem. Phys. Lipids 2000, 107, 191-220.
Drummond, C. J.; Fong, C., Surfactant self-assembly objects as novel drug delivery vehicles. Current Opinion in Colloid & Interface Science 1999, 4, 449-456.
Fong, W et al, Dynamic formation of nanostructured particles from vesicles via invertase hydrolysis for on-demand delivery, The Royal Society of Chemistry: Electronic Supplementary Material (ESI) for RSC Advances, 2016, S1-S22.
Hyde, S. T., Bicontinuous structures in lyotropic liquid crystals and crystalline hyperbolic surfaces. Current Opinion in Solid State and Materials Science 1996, 1, 653-662.
Israelachvili, J., The science and applications of emulsions—an overview. Colloids Surf. Physico chem. Eng. Aspects, 1994, 91, 1-8.
Kaasgaard, T.; Drummond, C. J., Ordered 2-D and 3-D nanostructured amphiphile self-assembly materials stable in excess solvent. Phys. Chem. Chem. Phys. 2006, 8, 4957-4975)
World Intellectual Property Office publication, WO 2014/179845.
International patent application nos PCT/AU2018/050366, PCT/AU2018/050367 and PCT/AU2018/050364.

It will be understood that the invention disclosed and defined in this specification extends to all alternative combinations of two or more of the individual features mentioned or evident from the text or drawings. All of these different combinations constitute various alternative aspects of the invention.

### EXAMPLES

### Example 1—Testing Mixtures of GMO and Excipients/Active Ingredients for Effect on Liquid Crystal Particle Size

GMO and excipient or active ingredient at various weight proportions were added into small HPLC vials. To ensure adequate mixing, the samples were initially heated above the melting temperature of GMO (>40° C.) and mixed vigorously with a metal spatula. The samples were then mixed via a roller mixer at ˜10 RPM at 40° C. for at least 3 days.

One week prior to SAXS experiment, 100 mg samples were loaded into a transparent polystyrene 96 well plate (Nunc™) and immersed in 200 μL of PBS buffer (pH 6.8). The samples were stored away from light at ambient temperature, to allow the samples to reach equilibrium.

Small Angle x-Ray Scattering (SAXS) Setup

The SAXS/WAXS beamline at Australian Synchrotron, Melbourne, Australia was used to determine the liquid crystalline nanostructure in the samples.

A custom-designed plate holder was used to mount the samples plate directly onto the SAXS/WAXS beamline. Scans were automated using a pre-loaded set of position variables based on the well positions within the plate, the exposure time was 5 sec.

Data were obtained at ambient temperature (˜22° C.). The experiments used a beam of wavelength λ=1.033 Å (12.0 keV) and a typical flux of 1.2×1013 photons/s. The 2-D diffraction images were recorded on a Pilatus 1M detector and radially integrated using the in-house software “ScatterBrain”.

The liquid crystal phase structures were determined by indexing the Bragg peaks according to their corresponding reflection laws (see Hyde, S. T., Bicontinuous structures in lyotropic liquid crystals and crystalline hyperbolic surfaces. Current Opinion in Solid State and Materials Science 1996, 1, 653-662).

The results in tables 1 to 3 below show GMO forming a bulk phase of complex crystalline particles (ie cubic or hexagonal or a mix of the two) following mixing with a number of ingredients and then emersion of the mix in a hydrophilic solvent (PBS buffer). When the ratio of ingredient to GMO increases, size of the lattice increases, which means the water channel size increases. The percentage w/w of the ingredient to GMO at which the water channel size increases is the “threshold” for the ingredient or the minimum amount of the ingredient required to increase the water channel size.

For some ingredients, further increases in the proportion of ingredient to GMO also resulted in a change in liquid crystalline structure to a structure with larger water channels eg a V2(Pn3m) to V2(Im3m) change. Other ingredients, did not increase the water channel size of the original liquid crystalline structure but at a threshold concentration did cause a change in liquid crystalline structure to a structure with larger water channels eg a V2(Pn3m) to V2(Im3m) change.

The inventors have also tested many ingredients that do not increase water channel size or result in a change in liquid crystalline structure.

Results

### 1.1 Ingredients Increasing Pn3m Lattice Size

TABLE 1
Tween 80 - V2(Pn3m) lattice size increased at 10-20%.
Tested in SBT126 and shown to increase Pn3m lattice
size from an ODT. A threshold of 8-10% or about 10%.
Transformation in structure by about 20%.

| Concentration | | |
|---|---|---|
| in GMO (%) | Phase structure | Lattice size |
| 1 | V2(Pn3m) | 91.9 |
| 5 | V2(Pn3m) | 97.9 |
| 10 | V2(Pn3m) | 106.3, 138.8 |
| 20 | V2(Pn3m) + V2(Im3m) | 137.5, 172.5 |

TABLE 2
Sodium cyclamate - V2(Pn3m) lattice size increased
at 5-10%. A threshold of 3-5% or about 5%.

| Concentration | | |
|---|---|---|
| in GMO (%) | Phase structure | Lattice size |
| 1 | V2(Pn3m) | 93.1 |
| 5 | V2(Pn3m) | 103.3 |
| 10 | V2(Pn3m) + other | 112.7 |

TABLE 3
Rosuvastatin - V2(Pn3m) lattice size increased at 5-15%.
A threshold of 3-5% or about 5%. Transformation in structure
by about 15%. Rosuvastatin was also tested in SBT177
and shown to increase Pn3m lattice size of liquid crystalline
particles self-assembled from an ODT.

| Concentration | | |
|---|---|---|
| in GMO (%) | Phase structure | Lattice size |
| 1 | V2(Pn3m) | 84.2 |
| 5 | V2(Pn3m) | 109.4 |
| 10 | V2(Pn3m) | 109.4, 130.7 |
| 15 | V2(Pn3m) + V2(Im3m) | 121.8, 148.1 |

TABLE 4
Saccharin sodium - V2(Pn3m) lattice size increased
at 5-10%. A threshold of 3-5% or about 5%.

| Concentration | | |
|---|---|---|
| in GMO (%) | Phase structure | Lattice size |
| 1 | V2(Pn3m) | 95.4 |
| 5 | V2(Pn3m) | 107.1 |
| 10 | V2(Pn3m) + others | 120.1 |

TABLE 5
Mg Trisilicate - V2(Pn3m) lattice size increased
at 35-50%. A threshold of 30-35% or about 35%.

| Concentration | | |
|---|---|---|
| in GMO (%) | Phase structure | Lattice size |
| 1 | V2(Pn3m) | 90.8 |
| 5 | V2(Pn3m) | 95.6 |
| 10 | V2(Pn3m) + H2? | 95.5 |
| 20 | V2(Pn3m) + H2? | 95.5, 55.6 |
| 35 | V2(Pn3m) + H2? | 114.2, 50.5 |
| 50 | V2(Pn3m) + H2? | 116.9, 50.7 |

TABLE 6
Ibuprofen lysine - V2(Pn3m) lattice size increased
at 5-10%. A threshold of 3-5% or about 5%.
Transformation in structure by about 10%.

| Concentration | | |
|---|---|---|
| in GMO (%) | Phase structure | Lattice size |
| 1 | V2(Pn3m) | 88.9 |
| 5 | V2(Pn3m) | 104.8 |
| 10 | V2(Pn3m) + V2(Ia3d) | 130.7, 189.4 |
| 15 | Lα + V2(Ia3d) | 80.69, 198.1 |

TABLE 7
Atorvastatin - V2(Pn3m) lattice size increased
at 10-35%. A threshold of 8-10% or about 10%.

| Concentration | | |
|---|---|---|
| in GMO (%) | Phase structure | Lattice size |
| 1 | V2(Pn3m) | 93.1 |
| 5 | V2(Pn3m) | 99.2 |
| 10 | V2(Pn3m) | 104.8 |
| 15 | V2(Pn3m) | 100.6 |
| 20 | V2(Pn3m) | 103.3 |
| 25 | V2(Pn3m) | 101.9 |
| 35 | V2(Pn3m) | 104.8 |

TABLE 8
SLS - V2(Pn3m) lattice size increased at 5-10%. A threshold of
3-5% or about 5%. SLS was also tested in SBT137 and shown to increase
Pn3m lattice size of liquid crystalline particles self-assembled
from an ODT. In ODT SBT125 - 5 mg SLS to 5.4 mg GMO or 48% SLS
(based on the weight of SLS and GMO) also caused a shift to Im3m
structure of 144-148 nm lattice size. Im3m is a cubic structure
with large water channels. Transition at about 50%.

| Concentration | | |
|---|---|---|
| in GMO (%) | Phase structure | Lattice size |
| 1 | V2(Pn3m) | 95.4 |
| 5 | V2(Pn3m) | 116.2 |
| 10 | V2(Pn3m) | 132.6 |

1.2 Ingredients Causing Transition to V2(Im3m) without Simple Water Channel Increase

Some ingredients caused transition to V2(Im3m) at certain concentrations relative to the GMO, without first expanding the V2(Pn3m) structure.

TABLE 9
Poloxamer 188 - transition to Im3m by 5%

| Concentration | | |
|---|---|---|
| in GMO (%) | Phase structure | Lattice size |
| 1 | V2(Pn3m) + V2(Im3m) | 91.9, 126.0 |
| 5 | V2(Im3m) | 128.1 |
| 10 | V2(Im3m) | 125.9 |

TABLE 10
Poloxamer 407 - transition to Im3m by 5%

| Concentration | | |
|---|---|---|
| in GMO (%) | Phase structure | Lattice size |
| 1 | V2(Pn3m) + V2(Im3m) | 92.0, 123.8 |
| 5 | V2(Im3m) | 123.8 |
| 10 | V2(Im3m) | 123.8 |
| 20 | V2(Im3m) + Lα | 123.8, 161.1 |

### 1.3 Ingredients Having No Effect on the Liquid Crystal Particle Size.

The tablet excipients Pharmaburst, crospovidone XL, sodium starch glycolate and sorbitol were also tested and found to have minimal impact on the GMO cubic structure formed.

Pharmaburst 1 to 50% in GMO resulted in V2(Pn3m) of 89-92 nm lattice size.

Crospovidone 1 to 50% in GMO resulted in V2(Pn3m) of 90-94 nm lattice size.

Sodium starch glycolate 1 to 50% in GMO resulted in V2(Pn3m) of 87-92 nm lattice size and some V2(Ia3d) of 131-141 nm at 5-20%.

Sorbitol 1 to 50% in GMO resulted in V2(Pn3m) of 83-92 nm lattice size (the small decrease in size possibly due to a dehydration effect) and at 1% some V2(Ia3d) blended with V2(Pn3m) of about 138 nm.

Silicon dioxide 1 to 10% in GMO resulted in V2(Pn3m) of 91-93 nm lattice size and some V2(Ia3d) of 138-145 nm blended with the V2(Pn3m) at 2.5-7.5%.

Magnesium stearate can self-assemble on its own. Some lamella phase was observed at 5-30% with a 50 nm lattice size. The GMO V2(Pn3m) was formed at 1-20% with a 93-95 nm lattice size and H2 formed at 30% with a 62 nm lattice size.

Sodium chloride was not tested but is expected not to impact lattice size.

Povidone (poly vinyl pyrrolidone) has been tested in blends of 3 to 40% by weight.

### Example 2—Formulations with 5:1 and 3:1 w/w Ratio of GMO to Enoxaparin (SBT265/SBT266) and 5:1 and 7:1 w/w Ratio of GMO to Dalteparin

Each tablet had 1 mg of active ingredient (ie enoxaparin sodium or dalteparin sodium). Neither tablet includes a water channel enhancer demonstrating that an enhancer is not required to formulate these larger active ingredients.

TABLE 11
5:1 and 3:1 w/w ratio of GMO to enoxaparin (SBT265/SBT266)

| Ingredient | SBT265 % w/w | SBT266 % w/w | Function |
|---|---|---|---|
| Pharmaburst - (co- | 69.87 | 71.91 | Filler, Taste masking, |
| processed mixture of | | | Disintegration agent. |
| Mannitol, Sorbitol | | | |
| Crospovidone & | | | |
| Silicon dioxide) | | | |
| Crospovidone XL | 9.88 | 9.88 | Disintegration agent |
| Sodium Chloride | 0.25 | 0.25 | Osmotic agent |
| Enoxaparin sodium | 1.02 | 1.02 | Drug substance/API |
| Povidone (Poly vinyl | 1.50 | 2.50 | Binder |
| pyrrolidone) | | | |
| Glyceryl Monooleate | 5.10 | 3.06 | Bio adhesive/ |
| (GMO) | | | Mucoadhesive agent, |
| | | | Gelling agent, nonionic |
| | | | surfactant, sustained |
| | | | release agent |
| Sodium Starch | 4.88 | 4.88 | Disintegration agent |
| Glycolate | | | |
| Colloidal Silicon | 1.50 | 1.50 | Glidant |
| Dioxide | | | |
| Magnesium Stearate | 1.00 | 1.00 | Lubricant |
| Ethanol | N/A * | N/A * | Solvent |

* Evaporated during the drying process.

TABLE 12
5:1 and 7:1 w/w ratio of GMO to dalteparin (SBT267/SBT268)

| Ingredient | SBT265 % w/w | SBT266 % w/w | Function |
|---|---|---|---|
| Pharmaburst - (co- | 69.87 | 80.95 | Filler, Taste masking, |
| processed mixture of | | | Disintegration agent. |
| Mannitol, Sorbitol | | | |
| Crospovidone & | | | |
| Silicon dioxide) | | | |
| Crospovidone XL | 9.83 | 10.50 | Disintegration agent |
| Sodium Chloride | 0.25 | 0.25 | Osmotic agent |
| Dalteparin sodium | 1.04 | 1.04 | Drug substance/API |
| Povidone (Poly vinyl | 1.50 | 1.75 | Binder |
| pyrrolidone) | | | |
| Glyceryl Monooleate | 5.18 | 7.25 | Bio adhesive/ |
| (GMO) | | | Mucoadhesive agent, |
| | | | Gelling agent, nonionic |
| | | | surfactant, sustained |
| | | | release agent |
| Sodium Starch | 4.83 | 5.50 | Disintegration agent |
| Glycolate | | | |
| Colloidal Silicon | 1.50 | 1.66 | Glidant |
| Dioxide | | | |
| Magnesium Stearate | 1.00 | 1.10 | Lubricant |
| Ethanol | N/A * | N/A * | Solvent |

* Evaporated during the drying process.

### Example 3—Manufacture of the Formulations in Example 2

Process for wet granulation manufacturing:
Enoxaparin sodium or dalteparin sodium was mixed with melted GMO
Povidone was dissolved in ethanol (granulation solution)
Pharmaburst, Crospovidone and Sodium chloride were granulated with the GMO:enoxaparin sodium or GMO:deltaparin sodium paste and the binder solution
The granules were dried and milled.
The milled granules were blended with the remaining excipients to form the final blend for compression.

The GMO used melted at about 35° C. Drying after the granulation step often occurred at 40° C., 5° C. above the GMO melting point.

### Example 3A—Manufacture of the Formulations in SBT176 (Table 22), SBT227, SBT226, SBT177 (Table 23), SBT233 (Table 23A), SBT 232, SBT122 (Table 14), SBT123 (Table 24)

Process for wet granulation manufacturing:
Rosuvastatin calcium was dispersed in melted GMO
Povidone, Menthol & Sacharin Sodium was dissolved in ethanol
Pharmaburst, Sodium chloride, Sodium cyclamate Crospovidone were granulated with the Rosuvastatin:GMO suspension and the Povidone solution
The granules were dried and milled.
The milled granules were blended with the remaining excipients to form the final blend for compression.

The GMO used melted at about 35° C. Drying after the granulation step often occurred at 40° C., 5° C. above the GMO melting point.

The tablets prepared by this method achieve 100% dissolution within 5 minutes (Dissolution apparatus II, paddles, 50 rpm, 900 ml, Citrate buffer pH 6.6).

Manufacture of the atorvastatin calcium trihydrate, oxycodone hydrochloride and epinephrine (adrenaline) ODTs also used this method.

### Example 4—Disintegration Time for the Formulations in Example 2

The formulations in Examples 2 were subjected to in vitro disintegration testing. Dissolution testing is not sufficiently precise for this purpose. An active ingredient can take about 5 minutes to be homogeneously dissolved in a 900 ml dissolution testing vessel, therefore, dissolution results taken earlier than 5 minutes are not reliable.

Disintegration testing was conducted in a basket-rack assembly and in accordance with Appendix XII A. Disintegration of the European Pharmacopoiea edition 9.0 (Ph. Eur. Method 2.9.1). The solvent was water at 37° C.

The enoxaparin sodium 5:1 formulation (SBT265) in Example 2 disintegrated within 25 to 40 seconds. The enoxaparin sodium 3:1 formulation (SBT266) in Example 2 disintegrated within 25 to 32 seconds.

The dalteparin sodium 5:1 formulation (SBT267) in Example 2 disintegrated within 30 to 50 seconds. The dalteparin sodium 7:1 formulation (SBT268) in Example 2 disintegrated within 35 to 80 seconds.

Without being bound by theory, it is thought that the larger size of the dalteparin sodium was responsible for the slightly slower disintegration of the dalteparin tablets, when compared to the enoxaparin tablets. The ODTs with more GMO to API were also slower to disintegrate.

### Example 5—Synchrotron Testing to Confirm Formation of Liquid Crystalline Particles from ODTs

Preparation of Samples Tablets for Simulated Dissolution Study

For equilibrium samples, the tablets were loaded into a transparent polystyrene 24 well plate (Nunc™) and immersed in PBS buffer (pH 6.8). Three different plates were tested. The first was hydrated the night before testing and stored away from light at ambient temperature overnight prior to SAXS experiment. The second was hydrated about 30 minutes before testing and the third hydrated 1½ to 2 hours before testing.

Small Angle x-Ray Scattering (SAXS) Setup

The SAXS/WAXS beamline at Australian Synchrotron, Melbourne, Australia was used to determine the liquid crystalline nanostructure in the samples.

A custom-designed plate holder was used to mount the samples plate directly onto the SAXS/WAXS beamline. Scans were automated using a pre-loaded set of position variables based on the well positions within the plate, the exposure time was 5 seconds.

Data were obtained at ambient temperature (˜22° C.). The experiments used a beam of wavelength λ=1.033 Å (12.0 keV) and a typical flux of 1.2×1013 photons/s. The 2-D diffraction images were recorded on a Pilatus 1M detector and radially integrated using the in-house software “ScatterBrain”.

The liquid crystal phase structures were determined by indexing the Bragg peaks according to their corresponding reflection laws (see Hyde, S. T., Bicontinuous structures in lyotropic liquid crystals and crystalline hyperbolic surfaces. Current Opinion in Solid State and Materials Science 1996, 1, 653-662).

Each sample was tested in triplicate at 125 times/locations. The results are in table 13 below.

TABLE 13
Results of synchrotron testing of hydrated ODTs from Example 2
Plate 1 - hydrated the night before synchrotron testing

| | |
|---|---|
| Control - 1 mg Rosuvastatin tablet with no GMO | Lalpha |
| 1 mg Daltaparin tablet (5:1) | Pm3m, Lalpha |
| 1 mg Daltaparin tablet (7:1) | Pm3m, Lalpha |
| 1 mg Enoxaparin tablet (5:1) | Pm3m, Lalpha |
| 1 mg Enoxaparin tablet (3:1) | Pm3m, Lalpha |

Plate 2 - hydrated 30 minutes before synchrotron testing

| | |
|---|---|
| Control - Rosuvastatin tablet with no GMO | Lalpha |
| Deltaparin tablet (5:1) | Pm3m, Lalpha |
| Deltaparin tablet (7:1) | Pm3m, Lalpha |
| Enoxaparin tablet (5:1) | Pm3m, Lalpha |
| Exnoxaparin tablet (3:1) | Pm3m, Lalpha |

Plate 3 - hydrated 1 to 1½ hours before synchrotron testing

| | |
|---|---|
| Control - Rosuvastatin tablet with no GMO | Lalpha |
| Daltaparin tablet (5:1) | Pm3m, Lalpha |
| Daltaparin tablet (7:1) | Pm3m, Lalpha |
| Enoxaparin tablet (5:1) | Pm3m, Lalpha |
| Enoxaparin tablet (3:1) | Pm3m, Lalpha |

Each tablet was tested in triplicate.

The lamella alpha phase for the control in A1 to A3 is expected to have been the result of the magnesium stearate, which forms lamella phase. All tablets with GMO present included the GMO cubic phase. Both the enoxaparin and daltaparin had Pn3m cubic phase with increased lattice size to above 110 nm (ie about 120 to 125 nm).

TABLE 13A
lattice and water channel size for dalteparin and enoxaparin

| Formulation | Pn3m lattice size (nm) | Water channel size (Å) |
|---|---|---|
| Dalteparin 5:1 | 121.6 | 59.09 |
| Dalteparin 7:1 | 119.9 | 57.76 |
| Enoxaparin 5:1 | 119.9 | 57.76 |
| Enoxaparin 3:1 | 125.2 | 61.91 |

### Example 6—Effect of Tween 80 on ODT

Using the process of Example 3 and the ingredients in Table 14 below, an ODT with and without Tween 80 has been developed. The GMO has retained the ability to form cubic liquid crystalline structures. SBT126 and SBT122 are the same 5 mg rosuvastatin formulations with a 1:1 ratio of rosuvastatin to GMO. However, SBT126 includes Tween 80 and has a Pn3m cubic lattice size of 143 nm (water channel size of 76 Å) rather than 123 nm. This indicates that Tween 80 can increase the size of water channels in liquid crystalline particles (ie a colloid dispersion) formed by contacting an ODT with a hydrophilic solvent.

TABLE 14
SBT122 and SBT126

| | % w/w for | % w/w for | |
|---|---|---|---|
| Ingredient | SBT122 | SBT126 | Function |
| Mannitol | 75.3 | 75.2 | Filler/Carrier |
| Rosuvastatin Calcium | 7.3 | 5.4 | Drug substance/API |
| Povidone (Poly vinyl | 1.7 | — | Binder |
| pyrrolidone) | | | |
| Glyceryl Monooleate | 7.3 | 5.4 | Bioadhesive/ |
| (GMO) | | | Mucoadhesive agent, |
| | | | Gelling agent, nonionic |
| | | | surfactant, sustained |
| | | | release agent |
| Sodium Starch | 5.0 | 5.0 | Disintegration agent |
| Glycolate | | | |
| Colloidal Silicon | 2.0 | 2.0 | Glidant |
| Dioxide | | | |
| Magnesium Stearate | 1.5 | 1.5 | Lubricant |
| Tween 80 | — | 2.3 | |
| Ethanol | N/A * | N/A * | Solvent |

* Evaporated during the drying process.

Disintegration testing was conducted in a basket-rack assembly and in accordance with Appendix XII A. Disintegration of the European Pharmacopoiea edition 9.0 (Ph. Eur. Method 2.9.1). The solvent was water at 37° C.

The SBT122 disintegrated within 8-10 minutes and achieved 100% dissolution in water within 15 minutes (Dissolution apparatus II, paddles, 50 rpm, 900 ml, Citrate buffer pH 6.6).

### Example 7—Effect of SLS on ODT

Using the process of Example 3 and the ingredients in Table 15 below, an ODT with and without SLS has been developed. The GMO has retained the ability to form cubic liquid crystalline structures. SBT130, SBT 136 and SBT137 are 5 mg rosuvastatin formulations with a 7:1 ratio of GMO to rosuvastatin. However, SBT136 includes SLS and SBT137 includes SLS as well as replacing mannitol with Pharmaburst. SBT 136 has a Im3m cubic lattice (lattice: 211.6 nm; water channels: 93.1 Å.) and SBT137 has a Pn3m cubic lattice size of 143 nm (water channels of 76 Å) rather than the SBT130 V2(Pn3m) cubic lattice size of 123 nm (water channels of 65 Å). This indicates that SLS can increase the size of water channels.

TABLE 15
SBT130, SBT136 and SBT137

| | % w/w for | % w/w for | % w/w for | |
|---|---|---|---|---|
| Ingredient | SBT130 | SBT136 | SBT137 | Function |
| Pharmaburst | — | — | 60.5 | Filler, Taste masking, |
| | | | | disintegration agent. |
| Mannitol | 61.0 | 60.5 | — | Filler/Carrier |
| Rosuvastatin Calcium | 1.6 | 1.6 | 1.6 | Drug substance/API |
| Povidone (Poly vinyl | 1.7 | 1.7 | 1.7 | Binder |
| pyrrolidone) | | | | |
| Glyceryl Monooleate | 10.5 | 10.5 | 10.5 | Bioadhesive/ |
| (GMO) | | | | Mucoadhesive agent, |
| | | | | Gelling agent, nonionic |
| | | | | surfactant, sustained |
| | | | | release agent |
| Sodium Starch | 20.0 | 20.0 | 20.0 | Disintegration agent |
| Glycolate | | | | |
| Colloidal Silicon | 2.0 | 1.5 | 1.5 | Glidant |
| Dioxide | | | | |
| Magnesium Stearate | 1.5 | 1.0 | 1.0 | Lubricant |
| SLS | — | 1.5 | 1.5 | Surfactant, |
| | | | | Penetration enhancer |
| Ethanol | N/A * | | N/A * | Solvent |

* Evaporated during the drying process.

Using the process of Example 3 and the ingredients in Table 16 below, an ODT with SLS has been developed (SBT125). SBT 125 has 5 mg SLS and 5.4 mg of GMO which is equivalent to 48% SLS. The Im3m lattice size identified at 10-60 minutes of testing was 145 nm. At 60-120 min Im3m structure was identified (lattice: 148 nm; water channels 54.3 Å).

TABLE 16
SBT125

| | % w/w for | |
|---|---|---|
| Ingredient | SBT125 | Function |
| Mannitol | 73.94 | Filler, carrier. |
| Rosuvastatin Calcium | 5.4 | Drug substance/API |
| Povidone (Poly vinyl | 1.7 | Binder |
| pyrrolidone) | | |
| Glyceryl Monooleate | 5.4 | Bio adhesive/ |
| (GMO) | | Mucoadhesive agent, |
| | | Gelling agent, nonionic |
| | | surfactant, sustained |
| | | release agent |
| Sodium Starch | 5.0 | Disintegration agent |
| Glycolate | | |
| Colloidal Silicon | 2.0 | Glidant |
| Dioxide | | |
| Magnesium Stearate | 1.5 | Lubricant |
| SLS | 5.0 | Surfactant, |
| | | Penetration enhancer. |
| Ethanol | N/A * | Solvent |

Using the process of Example 3 and the ingredients in Table 17 below, an ODT with SLS and Tartaric Acid has been developed (SBT134). SBT 134 has 5.0 mg SLS, 45 mg Tartaric acid and 36 mg of GMO. The Pn3m lattice size identified at 10-60 minutes of testing was 170 nm (97.57 Å).

TABLE 17
SBT134

| | % w/w for | |
|---|---|---|
| Ingredient | SBT134 | Function |
| Mannitol | 46.4 | Filler, carrier. |
| Rosuvastatin Calcium | 1.6 | Drug substance/API |
| Povidone (Poly vinyl | 1.7 | Binder |
| pyrrolidone) | | |
| Glyceryl Monooleate | 10.5 | Bio adhesive/ |
| (GMO) | | Mucoadhesive agent, |
| | | Gelling agent, nonionic |
| | | surfactant, sustained |
| | | release agent |
| Sodium Starch | 20.0 | Disintegration agent |
| Glycolate | | |
| Colloidal Silicon | 2.0 | Glidant |
| Dioxide | | |
| Magnesium Stearate | 1.5 | Lubricant |
| SLS | 1.5 | Surfactant, |
| | | Penetration enhancer. |
| Tartaric acid | 13.2 | Acidifying agent |
| Ethanol | N/A * | Solvent |

### Example 8—Effect of Rosuvastatin on ODT

Using the process of Example 3 and the ingredients in Table 18 below, an ODT with and without rosuvastatin has been developed. The GMO has retained the ability to form cubic liquid crystalline structures. SBT177 has 5 mg rosuvastatin formulations with a 4:1 ratio of GMO to rosuvastatin. SBT189 is a placebo of SBT176 ie it has 5 mg GMO but no rosuvastatin present. However, SBT177 has a Pn3m cubic lattice size of 143 nm (76 Å) rather than the 126 nm (water channels of 62 Å) for the placebo. This indicates that rosuvastatin can increase the size of water channels.

Using the process of Example 3 and the ingredients in Table 23 below, an ODT with 20% rosuvastatin to GMO has been developed.

TABLE 18
SBT177 and SBT189

| | % w/w for | % w/w for | |
|---|---|---|---|
| Ingredient | SBT177 | SBT189 | Function |
| Pharmaburst - (co- | 64.5 | 74.11 | Filler, Taste masking, |
| processed mixture of | | | Disintegration agent. |
| Mannitol, Sorbitol | | | |
| Crospovidone & | | | |
| Silicon dioxide) | | | |
| Crospovidone XL | 15.0 | | Disintegration agent |
| Sodium Chloride | 0.3 | 0.25 | Osmotic agent |
| Sodium Cyclamate | 0.6 | 0.60 | Sweetener |
| Saccharin Sodium | 0.4 | 0.4 | Sweetener |
| Menthol | 0.2 | 0.2 | Flavouring agent |
| Rosuvastatin Calcium | 1.8 | — | Drug substance/API |
| Povidone (Poly vinyl | 2.5 | 1.5 | Binder |
| pyrrolidone) | | | |
| Glyceryl Monooleate | 7.3 | 5.4 | Bio adhesive/ |
| (GMO) | | | Mucoadhesive agent, |
| | | | Gelling agent, nonionic |
| | | | surfactant, sustained |
| | | | release agent |
| Sodium Starch | 5.0 | 5.0 | Disintegration agent |
| Glycolate | | | |
| Colloidal Silicon | 1.5 | 1.5 | Glidant |
| Dioxide | | | |
| Magnesium Stearate | 1.0 | 1.0 | Lubricant |
| Ethanol | N/A * | N/A * | Solvent |

* Evaporated during the drying process.

SBT177 preparation is according to Example 3 except mixing of the rosuvastatin calcium and GMO is for 1-2 minutes.

The SBT177 tablets weighed 300 mg, had a hardness of 2.4-3.5 kp, friability of 0.1%, 2.65 mm thickness and disintegrated in 60-90 seconds following emersion in a hydrophilic solvent.

The ODTs of Table 18 achieve 100% dissolution within 5 minutes (Dissolution apparatus II, paddles, 50 rpm, 900 ml, Citrate buffer pH 6.6).

The tablets were administered sublingually to three different human subjects (one Caucasian male, one Caucasian female and one Asian male) and the speed of tablet disintegration monitored. The SBT177 disintegrated within 20 to 40 seconds of administration for all three subjects.

The manufacturing of tablets involved mixing of Rosuvastatin with the GMO at its melting point for a short period, until a homogenous dispersion was obtained (approximately 5 minutes) and then mixed with other excipients using a high shear mixer. When combined with the other excipients the temperature of GMO returned to below the GMO melting point and the GMO returned to its semi-solid form.

The formulation of SBT122 disintegrated within 20-40 seconds of contact with oral mucosa (Basket-rack assembly, Ph. Eur. Method 2.9.1, water at 37° C.). The formulation of Table 4 disintegrated within 40-90 seconds of contact with oral mucosa.

12 mm round tablets of this formulation were stability tested at 5° C. for 6 months. Assay of the 5 mg rosuvastatin showed 100.1% at t=0, 100.4% at t=3 months and 97.9% at t=6 months. The formulation was also stability tested at 25° C./60% RH for 6 months. Assay of the 5 mg rosuvastatin showed 100.1% at t=0, 98.6% at t=3 months and 97.6% at t=6 months. In addition, the assay of the tablets showed 0.05% at t=0, 0.31% at t=3 months and 0.49% at t=6 months of rosuvastatin in the lactone form and 0.3% at t=0, 0.31% at t=3 months and 0.36% at t=6 months of 5-oxo-rosuvastatin calcium (TP-13 impurity 1) at following storage at 25° C./60% RH. The assay of the tablets also showed 0.05% at t=0, 0.08% at t=3 months and 0.05% at t=6 months of rosuvastatin in the lactone form and 0.3% at t=0, 0.23% at t=3 months and 0.24% at t=6 months of the 5-oxo-rosuvastatin calcium at both 3 and 6 months at 5° C.

### Example 9—Effect of Sodium Bicarbonate on ODT

Using the process of Example 3 and the ingredients in Table 19 below, an ODT with sodium bicarbonate has been developed (SBT127). SBT 127 has 5 mg Sodium carbonate and 5.4 mg of GMO which is equivalent to 48% Sodium Carbonate. The Pn3m lattice size identified at 10-60 minutes of testing was 175.9 nm (101.6 Å water channel).

TABLE 19
SBT127

| | % w/w for | |
|---|---|---|
| Ingredient | SBT127 | Function |
| Mannitol | 73.9 | Filler, Carrier |
| Rosuvastatin Calcium | 5.4 | Drug substance/API |
| Povidone (Poly vinyl | 1.7 | Binder |
| pyrrolidone) | | |
| Glyceryl Monooleate | 5.4 | Bio adhesive/ |
| (GMO) | | Mucoadhesive agent, |
| | | Gelling agent, nonionic |
| | | surfactant, sustained |
| | | release agent |
| Sodium Starch | 5.0 | Disintegration agent |
| Glycolate | | |
| Colloidal Silicon | 2.0 | Glidant |
| Dioxide | | |
| Magnesium Stearate | 1.0 | Lubricant |
| Sodium bicarbonate | 4.7 | Alkalizing/Basifying agent |
| Ethanol | N/A * | Solvent |

### Example 10—Effect of Menthol on ODT

Using the process of Example 3 and the ingredients in Table 20 below, an ODT with sodium bicarbonate has been developed (SBT131). SBT 131 has 4 mg menthol and 5.4 mg of GMO which is equivalent to 42.5% menthol. The Pn3m lattice size identified at 10-60 minutes of testing was 141 nm.

TABLE 20
SBT131

| | % w/w for | |
|---|---|---|
| Ingredient | SBT131 | Function |
| Mannitol | 74.9 | Filler, Carrier |
| Menthol | 3.5 | Flavouring agent |
| Rosuvastatin Calcium | 5.4 | Drug substance/API |
| Povidone (Poly vinyl | 1.7 | Binder |
| pyrrolidone) | | |
| Glyceryl Monooleate | 5.44 | Bio adhesive/ |
| (GMO) | | Mucoadhesive agent, |
| | | Gelling agent, nonionic |
| | | surfactant, sustained |
| | | release agent |
| Sodium Starch | 3.5 | Disintegration agent |
| Glycolate | | |
| Colloidal Silicon | 5.4 | Glidant |
| Dioxide | | |
| Magnesium Stearate | 1.7 | Lubricant |
| Menthol | 3.5 | Flavouring agent |
| Ethanol | N/A * | Solvent |

### Example 11—In Vitro Release Testing

Release of an active ingredient from and ODT through a mucosal membrane can be tested in vitro.

Porcine buccal mucosa was freshly isolated from pigs cheeks, mounted between modified Ussing chambers with a donor chamber, receptor chamber and the porcine buccal mucosa in between with a diffusional area of 0.64 cm², and incubated in Krebs bicarbonate Ringer buffer (KBR, pH 7.4) for 30 min. The tablet was applied to the porcine buccal mucosa (ie in the donor chamber) and, when necessary, Parafilm was applied to cover the formulation (ie for tablets and for mixtures containing glyceryl monooleate (GMO) and rosuvastatin). The Parafilm prevented the various formulations from detaching from the buccal mucosa. KBR buffer (1.5 mL) was then added to both the donor and receptor chambers, and receptor samples (200 μL) were collected from the receptor chamber at various time points up to 4-5 hours to determine the amount of rosuvastatin that passed through the porcine buccal mucosa to the receptor chamber. 200 μL of fresh KBR was dispensed into the receptor chamber after each collection (to ensure volume balance). Receptor chamber samples were quantified by HPLC.

Positive control was tested by making solutions of 0.4 and 0.8 mg/1.5 ml active ingredient in KBR solution, equivalent to 1:1 and 7:1 ODT's.

Rosuvastatin Release Testing

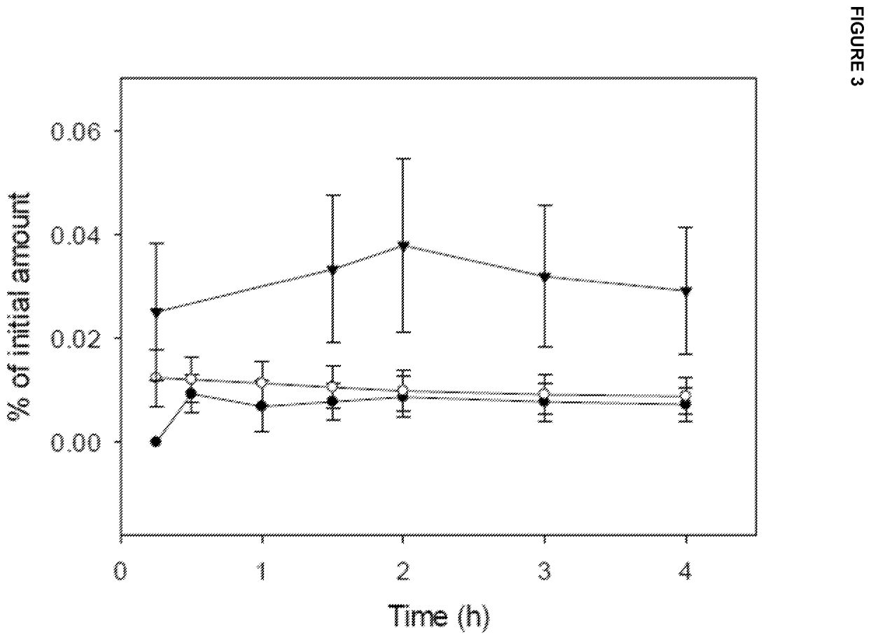

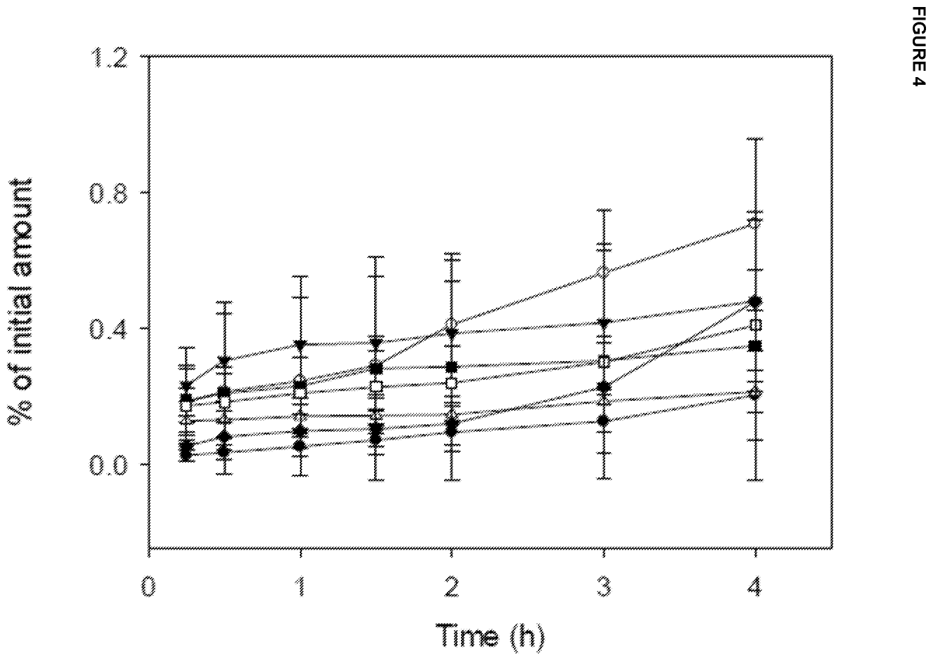

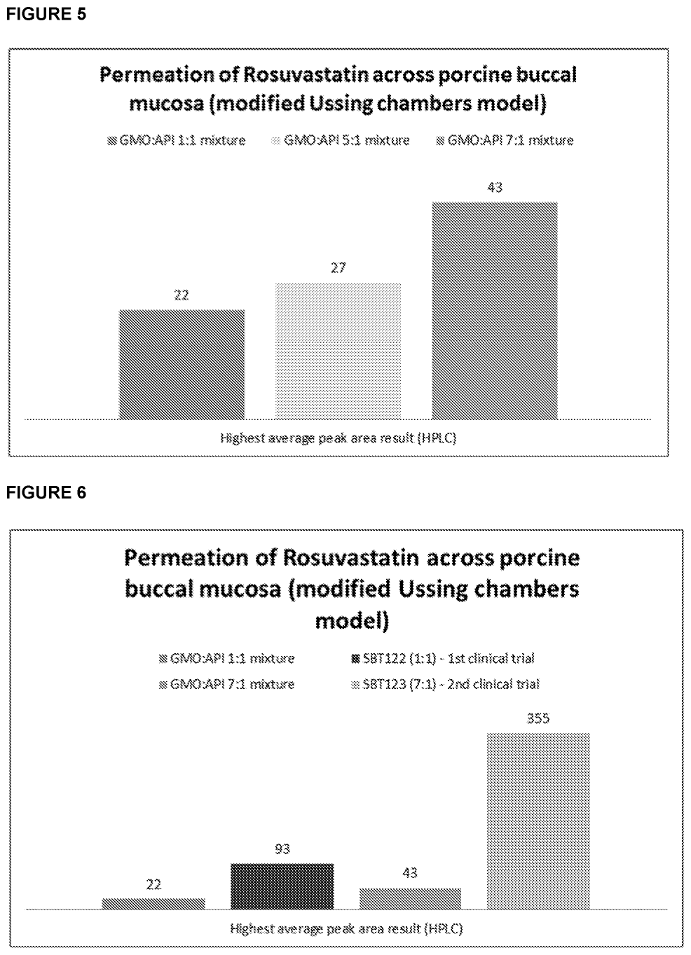

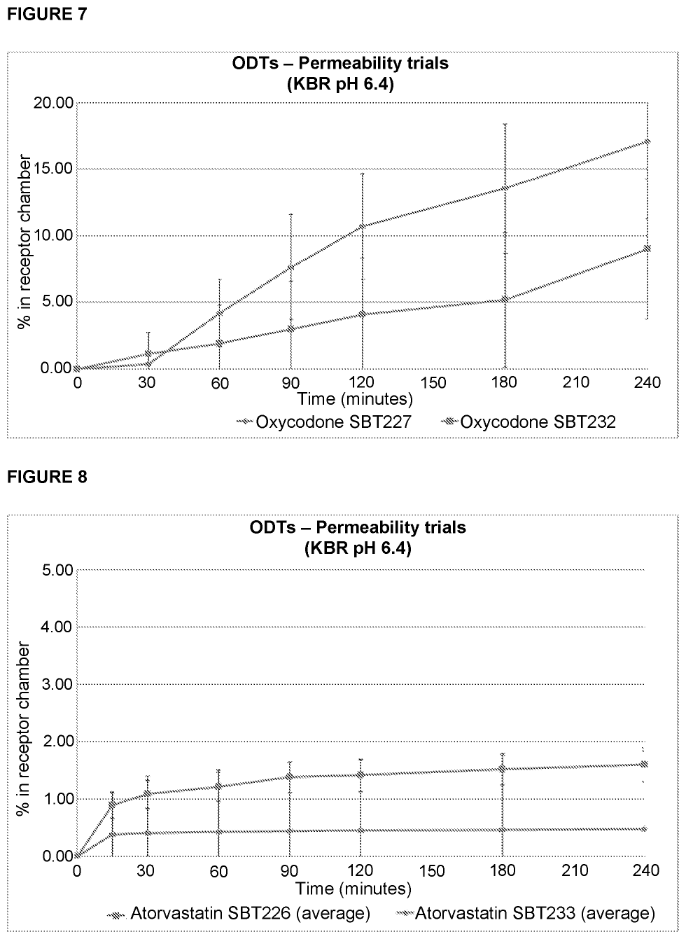

When a mixture was tested, GMO and rosuvastatin were manually mixed on the day of the experiment. The amount of rosuvastatin in the receptor chamber is in FIG. 3. The highest average peak area is in Table 21 below.

TABLE 21
effect of ratio of GMO to API

| | Highest average peak |
|---|---|
| Formulation | area result (HPLC) |
| GMO:API 1:1 mixture | 22 |
| GMO:API 5:1 mixture | 27 |
| GMO:API 7:1 mixture | 43 |

These results show that including more GMO than API improves the quantity of permeation of the API through the buccal mucosa.

When the tablets were tested, the tablet of Table 22 was applied to the porcine buccal mucosa whole but the tablet of Table 23 was halved before application to the mucosa to better enable the parafilm to hold the tablet in place. The amount of rosuvastatin in the receptor chamber during testing of the tablets is in FIG. 4.

TABLE 22
ODT with 1:1 GMO to statin (SBT176)

| Ingredient | % w/w | Function |
|---|---|---|
| Pharmaburst - (co- | 68.67 | Filler, Taste masking, |
| processed mixture of | | Disintegration agent. |
| Mannitol, Sorbitol | | |
| Crospovidone & | | |
| Silicon dioxide) | | |
| Crospovidone XL | 15.00 | Disintegration agent |
| Sodium Chloride | 0.25 | Osmotic agent |
| Sodium Cyclamate | 0.60 | Sweetener |
| Saccharin Sodium | 0.40 | Sweetener |
| Menthol (Optional) | 0.20 | Flavouring agent |
| Rosuvastatin Calcium | 5.44 | Drug substance/API |
| (micronized) | | |
| Povidone (Poly vinyl | 1.50 | Binder |
| pyrrolidone) | | |
| Glyceryl Monooleate | 5.44 | Bio adhesive/ |
| (GMO) | | Mucoadhesive agent, |
| | | Gelling agent, nonionic |
| | | surfactant, sustained |
| | | release agent |
| Sodium Starch | 5.00 | Disintegration agent |
| Glycolate | | |
| Colloidal Silicon | 1.50 | Glidant |
| Dioxide | | |
| Magnesium Stearate | 1.00 | Lubricant |
| Ethanol | N/A * | Solvent |

* Evaporated during the drying process.

A further ODT (SBT227) was prepared using 5% w/w of oxycodone hydrochloride and the same amount of GMO. The crospovidone was also reduced to 10% w/w and the Pharmaburst increased to 69.55% w/w. The ODT (SBT226) had a 1:1 ratio of GMO to oxycodone hydrochloride and 5 mg oxycodone hydrochloride.

TABLE 23
ODT with 4:1 GMO to statin (SBT177)

| Ingredient | % w/w | Function |
|---|---|---|
| Pharmaburst - (co- | 64.47 | Filler, Taste masking, |
| processed | | Disintegration agent. |
| mixture of | | |
| Mannitol, Sorbitol | | |
| Crospovidone & | | |
| Silicon dioxide) | | |
| Crospovidone XL | 15.00 | Disintegration agent |
| Sodium Chloride | 0.25 | Osmotic agent |
| Sodium Cyclamate | 0.60 | Sweetener |
| Saccharin Sodium | 0.40 | Sweetener |
| Menthol | 0.20 | Flavouring agent |
| Rosuvastatin Calcium | 1.81 | Drug substance/API |
| (micronized) | | |
| Povidone (Poly vinyl | 2.50 | Binder |
| pyrrolidone) | | |
| Glyceryl Monooleate | 7.26 | Bio adhesive/ |
| (GMO) | | Mucoadhesive agent, |
| | | Gelling agent, nonionic |
| | | surfactant, sustained |
| | | release agent |
| Sodium Starch | 5.00 | Disintegration agent |
| Glycolate | | |
| Colloidal Silicon | 1.50 | Glidant |
| Dioxide | | |
| Magnesium Stearate | 1.00 | Lubricant |
| Ethanol | N/A * | Solvent |

* Evaporated during the drying process.

A further ODT (SBT233) containing 10 mg atorvastatin was prepared with a 4:1 ratio of GMO to atorvastatin. The formulation is in Table 23A below.

TABLE 23A
ODT with 4:1 GMO to atorvastatin (SBT233)

| Ingredient | % w/w | Function |
|---|---|---|
| Pharmaburst - (co- | 58.86 | Filler, Taste masking, |
| processed mixture of | | Disintegration agent. |
| Mannitol, Sorbitol | | |
| Crospovidone & | | |
| Silicon | | |
| dioxide) | | |
| Crospovidone XL | 13.82 | Disintegration agent |
| Sodium Chloride | 0.22 | Osmotic agent |
| Sodium Cyclamate | 0.53 | Sweetener |
| Saccharin Sodium | 0.41 | Sweetener |
| Menthol | 0.21 | Flavouring agent |
| Atorvastatin Calcium | 3.19 | Drug substance/API |
| Trihydrate | | |
| Povidone (Poly vinyl | 2.50 | Binder |
| pyrrolidone) | | |
| Glyceryl Monooleate | 12.76 | Bio adhesive/ |
| (GMO) | | Mucoadhesive agent, |
| | | Gelling agent, nonionic |
| | | surfactant, sustained |
| | | release agent |
| Sodium Starch | 5.00 | Disintegration agent |
| Glycolate | | |
| Colloidal Silicon | 1.50 | Glidant |
| Dioxide | | |
| Magnesium Stearate | 1.00 | Lubricant |
| Ethanol | N/A * | Solvent |

A further ODT (SBT232) was prepared in accordance with Table 23A but using 1.67% w/w of oxycodone hydrochloride and four times as much GMO (6.67% w/w). The crospovidone was also reduced to 10% w/w and the Pharmaburst increased to 65.22% w/w, when compared to the rosuvastatin formulation in Table 23A. The ODT (SBT232) had a 4:1 ratio of GMO to oxycodone hydrochloride and 5 mg oxycodone hydrochloride.

TABLE 24
SBT123

| Ingredient | % w/w | Function |
|---|---|---|
| Mannitol | 76.0 | Filler/Carrier |
| Rosuvastatin Calcium | 1.6 | Drug substance/API |
| (micronized) | | |
| Povidone (Poly vinyl | 3.4 | Binder |
| pyrrolidone) | | |
| Glyceryl Monooleate | 10.5 | Bioadhesive/ |
| (GMO) | | Mucoadhesive agent, |
| | | Gelling agent, nonionic |
| | | surfactant, sustained |
| | | release agent |
| Sodium Starch | 5.0 | Disintegration agent |
| Glycolate | | |
| Colloidal Silicon | 2.0 | Glidant |
| Dioxide | | |
| Magnesium Stearate | 1.5 | Lubricant |
| Ethanol | N/A * | Solvent |

TABLE 25
effect of tablet formulation

| | Highest average peak |
|---|---|
| Formulation | area result (HPLC) |
| GMO:API 1:1 mixture | 22 |
| GMO:API 7:1 mixture | 43 |
| Formulation of Table 14 | 93 |
| (1:1 ratio) (SBT122) | |
| Formulation of Table 24 | 355 |
| (7:1 ratio) (SBT123) | |

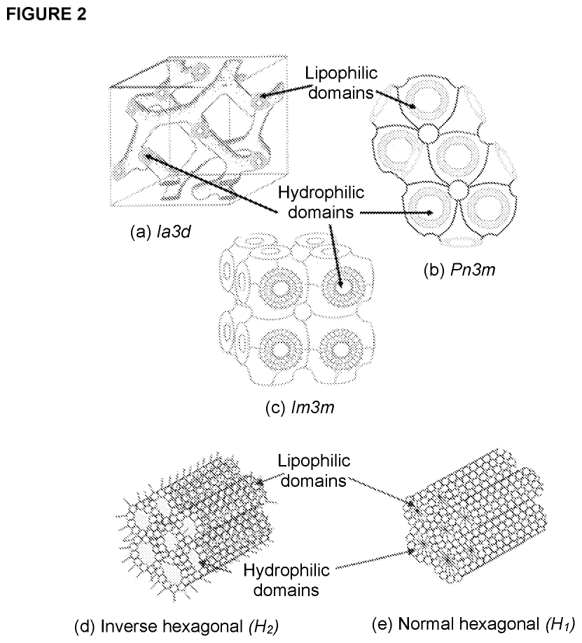

The results of Table 25 show that permeation through the buccal mucosa is better with a tablet than with a simple mixture. Also, an increased ratio of GMO to API still improves permeation in the tablet form. The results are shown in FIGS. 1 to 4.

When tested in the permeability cell, SL tablets were well hydrated and disintegrated within the experimental timeframe. 0.17% or 1.34 ng/ml/min of rosuvastatin in the 1:1 SL tablet used in the clinical trial achieving 125-132% (GS & VO) of the oral Crestor tablet, passed into the receiving chamber. Interestingly 1.41% or 6.1 ng/ml/min more of the 7:1 rosuvastatin passed into the receiving chamber.

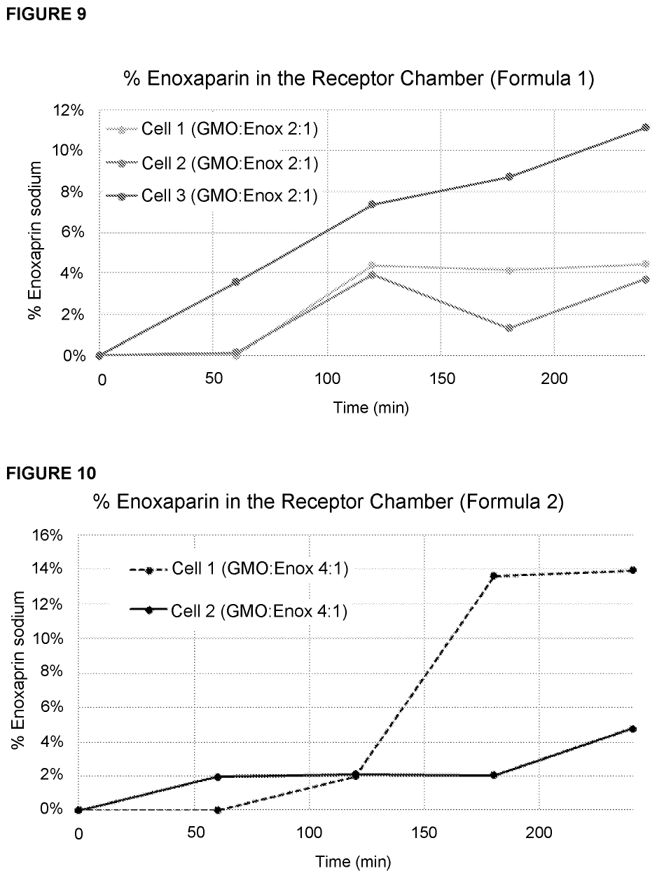

The appearance of oxycodone in the receptor chamber over time is depicted in FIG. 9 after application of ODT SBT227 (diamond) and ODT SBT232 (square) to porcine buccal mucosa in the donor chamber of the Ussing chamber. Data are presented as mean±SEM (n=5). Both formulations demonstrate slow release characteristics. SBT227, with a 1:1 ratio of active ingredient to GMO, released the API relatively faster compared to SBT232, with a 1:4 ratio of active ingredient to GMO. Without being bound by theory, this is thought to be due to the lower quantity of GMO in the SBT227 formulation.

The appearance of atorvastatin in the receptor chamber over time is depicted in FIG. 10 after application of ODT SBT226 (square) and SBT233 (diamond) to porcine buccal mucosa in the donor chamber of the Ussing chamber. Data are presented as mean±SEM (n=5). Both formulations demonstrate slow release characteristics. SBT226, with a 1:1 ratio of active ingredient to GMO, released the API relatively faster comparing to SBT233, with a 1:4 ratio of active ingredient to GMO. Without being bound by theory, this is thought to be due to the lower quantity of GMO in the SBT226 formulation.

Adrenaline degrades very quickly upon exposure to air or light. The in vitro release of adrenaline has not yet been tested as a suitable method accounting for the instability of adrenaline needs to be developed. However, in view of the results to date, adrenaline is expected to pass through the porcine oral mucosa in a Ussing chamber. The permeation of the active ingredient from the ODT of the invention was tested for ODTs containing oxycodone and ODTs containing atorvastatin to establish that the ODT functioned to deliver active ingredients having varied Log P values and varied dosages. This time samples were taken from the receiving chamber of the Ussing chamber repeatedly at 0.5, 1, 1.5, 2, 3 and 4 hours to establish not only that the active ingredient permeated the mucosa but that release of the active ingredient was prolonged.

Enoxaparin In Vitro Release Testing

KBR buffer (pH 7.4) was used for enoxaparin in vitro release testing. It was prepared according to Table 26.

TABLE 26
KBR buffer components

| Compound | mM | mOsm |
|---|---|---|
| NaCl | 115.385 | 230.769 |
| KCl | 4.155 | 8.311 |
| NaHCO₃ | 21.905 | 43.810 |
| Glucose | 12.209 | 12.209 |
| Hepes | 3.987 | 3.987 |
| MgSO₄•7H₂O | 1.163 | 2.325 |
| NaH₂PO₄•2H₂O | 1.596 | 3.192 |
| CaCl₂•2H₂O | 2.503 | 7.510 |
| | Total mOsm | 304.602 |

In the standard Ussing chambers, pig cheek skin was mounted in between the donor and receptor chambers. The porcine buccal mucosa had a diffusional area of 0.64 cm², and was incubated in Krebs bicarbonate Ringer buffer (KBR, pH 7.4) for 30 min. In the modified Ussing Chamber model, enoxaparin and GMO solutions were added to the donor chamber to test if and how much drug would permeate the membrane. Tris or KBR buffer (1.5 mL) was then added to both the donor and receptor chambers, and receptor samples (200 μL) were collected from the receptor chamber at various time points up to 4 hours to determine the amount of enoxaparin that passed through the membrane to the receptor chamber. 200 μL of fresh KBR was dispensed into the receptor chamber after each collection (to ensure volume balance). Receptor chamber samples were quantified by activity-based quantification kits. Test kits from 5-Diagnostics determined the anti-coagulant efficacy of the enoxaparin. Specifically anti-clotting factors II and X were tested. Briefly, frozen samples from each time point were defrosted and shaken gently to ensure homogeneous dispersion. They were then analysed using a 96-well flat bottom well ELISA plate pre-warmed at 37° C. and a Perkin Elmer Ensight Multi-mode plate reader set at an absorbance of 405 nm and at room temperature, with sample absorbance compared to a calibration curve prepared from standards of enoxaparin at pH 7.4.

The results for the blends were as below:

TABLE 27
Starting concentrations in each cell

| | |
|---|---|
| Formula 1 (GMO:Enoxaparin 2:1) | Cell 1 - Total mix concentration: 0.1341 g/1.5 ml |
| | Cell 2 - Total mix concentration: 0.0903 g/1.5 ml |
| | Cell 3 - Total mix concentration: 0.0935 g/1.5 ml |
| Formula 2 (GMO:Enoxaparin 4:1) | Cell 1 - Total mix concentration: 0.0601 g/1.5 ml |
| | Cell 2 - Total mix concentration: 0.0686 g/1.5 ml |
| | Cell 3 - Total mix concentration: 0.0631 g/1.5 ml |

### Formula 1:

TABLE 28
Concentration (mg/mL) in receptor chamber
at various time points for Formula 1

| | 1 hr | | 2 hr | | 3 hr | | 4 hr | |
|---|---|---|---|---|---|---|---|---|
| | Aver- | | Aver- | | Aver- | | Aver- | |
| | age | Stdev | age | Stdev | age | Stdev | age | Stdev |
| Cell 1 | 0.000 | 0.000 | 3.930 | 0.156 | 3.190 | 0.235 | 3.485 | 0.205 |
| Cell 2 | 0.085 | 0.007 | 2.350 | 0.217 | 0.475 | 0.078 | 2.125 | 0.177 |
| Cell 3 | 2.210 | 0.000 | 4.305 | 0.191 | 4.813 | 1.167 | 6.220 | 0.636 |

TABLE 29
Mass (mg) in receptor chamber based on total volume (1.5
mL) in the receptor chamber (mg/mL × 1.5 mL) for Formula 1

| | 1 hr | | 2 hr | | 3 hr | | 4 hr | |
|---|---|---|---|---|---|---|---|---|
| | Aver- | | Aver- | | Aver- | | Aver- | |
| | age | Stdev | age | Stdev | age | Stdev | age | Stdev |
| Cell 1 | 0.000 | 0.000 | 5.895 | 0.233 | 4.485 | 0.353 | 5.228 | 0.308 |
| Cell 2 | 0.128 | 0.011 | 3.525 | 0.325 | 0.713 | 0.117 | 3.188 | 0.265 |
| Cell 3 | 3.315 | 0.000 | 6.458 | 0.286 | 7.220 | 1.750 | 9.330 | 0.955 |

TABLE 30
Adjusted mass (mg) in receptor chamber, correcting for
decrease in mass due to sampling (0.2 mL out of 1.5
mL) at each time point (Mass from previous time point
× 0.2/1.5 + current measured mass) for Formula 1

| | 1 hr | | 2 hr | | 3 hr | | 4 hr | |
|---|---|---|---|---|---|---|---|---|
| | Aver- | | Aver- | | Aver- | | Aver- | |
| | age | Stdev | age | Stdev | age | Stdev | age | Stdev |
| Cell 1 | 0.000 | 0.000 | 5.895 | 0.233 | 5.571 | 0.353 | 5.970 | 0.308 |
| Cell 2 | 0.128 | 0.011 | 3.542 | 0.325 | 1.185 | 0.117 | 3.345 | 0.265 |
| Cell 3 | 3.315 | 0.000 | 6.900 | 0.286 | 8.140 | 1.750 | 10.415 | 0.955 |

TABLE 31
Percentage of enoxaparin in receptor chamber based upon the total enoxaparin sodium
added to the donor chamber at the start of the experiment for Formula 1

| | 1 hr | | 2 hr | | 3 hr | | 4 hr | |
|---|---|---|---|---|---|---|---|---|
| | Aver- | | Aver- | | Aver- | | Aver- | |
| | age | Stdev | age | Stdev | age | Stdev | age | Stdev |
| Cell 1 | 0.00% | 0.00% | 4.40% | 0.17% | 4.15% | 0.26% | 4.45% | 0.23% |
| Cell 2 | 0.14% | 0.01% | 3.92% | 0.36% | 1.31% | 0.13% | 3.70% | 0.29% |
| Cell 3 | 3.55% | 0.00% | 7.38% | 0.31% | 8.71% | 1.87% | 11.14% | 1.02% |

The results of Table 31 are plotted in FIG. 9.
Repeated analysis of the results shown in Tables 28-31 indicated that the concentrations observed in cell 2 at 2 h and 3 h were real and reproducible results.
For Formula 1, 4-11% of total Enoxaparin sodium in the donor chamber was measured in the receptor chamber after 4 hours indicating the release and permeability of drug through the membrane.

### Formula 2:

TABLE 32
Concentration (mg/mL) in receptor chamber
at various time points for Formula 2

| | 1 hr | | 2 hr | | 3 hr | | 4 hr | |
|---|---|---|---|---|---|---|---|---|
| | Aver- | | Aver- | | Aver- | | Aver- | |
| | age | Stdev | age | Stdev | age | Stdev | age | Stdev |
| Cell 1 | 0.000 | 0.000 | 0.798 | 0.145 | 5.343 | 0.430 | 4.850 | 0.014 |
| Cell 2 | 0.900 | 0.099 | 0.840 | 0.151 | 0.808 | 0.056 | 2.050 | 0.071 |

TABLE 33
Mass (mg) in receptor chamber based on total volume (1.5
mL) in the receptor chamber (mg/mL × 1.5 mL) for Formula 2

| | 1 hr | | 2 hr | | 3 hr | | 4 hr | |
|---|---|---|---|---|---|---|---|---|
| | Aver- | | Aver- | | Aver- | | Aver- | |
| | age | Stdev | age | Stdev | age | Stdev | age | Stdev |
| Cell 1 | 0.000 | 0.000 | 1.196 | 0.218 | 8.015 | 0.646 | 7.275 | 0.021 |
| Cell 2 | 1.350 | 0.148 | 1.260 | 0.227 | 1.211 | 0.083 | 3.075 | 0.106 |

TABLE 34
Adjusted mass (mg) in receptor chamber, correcting for
decrease in mass due to sampling (0.2 mL out of 1.5
mL) at each time point (Mass from previous time point
× 0.2/1.5 + current measured mass) for Formula 2

| | 1 hr | | 2 hr | | 3 hr | | 4 hr | |
|---|---|---|---|---|---|---|---|---|
| | Aver- | | Aver- | | Aver- | | Aver- | |
| | age | Stdev | age | Stdev | age | Stdev | age | Stdev |
| Cell 1 | 0.000 | 0.000 | 1.196 | 0.218 | 8.175 | 0.646 | 8.365 | 0.021 |
| Cell 2 | 1.350 | 0.148 | 1.440 | 0.227 | 1.403 | 0.083 | 3.262 | 0.106 |

TABLE 35
Percentage of enoxaparin in receptor chamber based upon the total enoxaparin sodium
added to the donor chamber at the start of the experiment for Formula 2

| | 1 hr | | 2 hr | | 3 hr | | 4 hr | |
|---|---|---|---|---|---|---|---|---|
| | Aver- | | Aver- | | Aver- | | Aver- | |
| | age | Stdev | age | Stdev | age | Stdev | age | Stdev |
| Cell 1 | 0.00% | 0.00% | 1.99% | 0.36% | 13.60% | 1.07% | 13.92% | 0.04% |
| Cell 2 | 1.97% | 0.22% | 2.10% | 0.33% | 2.05% | 0.12% | 4.76% | 0.15% |

The results of Table 35 are plotted in FIG. 10.
After 4 hours, between 4-14% of the Enoxaparin sodium was found in the receptor chamber.
For Formula 2, about 5-14% of total Enoxaparin sodium in the donor chamber was measured in the receptor chamber after 4 hours indicating the release and permeability of drug through the membrane.
Results for both Formula 1 and Formula 2 indicate that a substantial amount of enoxaparin is able to cross the membrane with the GMO enhancer.

1. An oral disintegrating tablet, suitable for administration to the oral mucosa, comprising
an amphiphilic compound capable of self-assembling into liquid crystalline particles when contacted by a hydrophilic solvent;
a therapeutically effective amount of a 1 kDa to 10 kDa active ingredient; and
a pharmaceutically acceptable disintegrant.

2. The tablet according to claim 1, wherein the active ingredient is a low molecular weight heparin.

3. The tablet according to claim 1, wherein the active ingredient is ardeparin, bemiparin, nadroparin, reviparin, enoxaparin, parnaparin, certoprain, dalteparin and/or tinzaparin, preferably enoxaparin and/or dalteparin.

4. The tablet according to claim 1, further comprising a water channel enhancer.

5. The tablet according to claim 4, wherein the water channel enhancer increases the size of the water channels in the liquid crystalline structure formed when the amphiphilic compound self-assembles by at least about 5 Å.

6. The tablet according to claim 4, wherein the water channel enhancer increases the lattice size in the liquid crystalline structure formed when the amphiphilic compound self-assembles by at least about 10 nm.

7. The tablet according to claim 6, wherein the water channel enhancer increases the lattice size in the liquid crystalline structure formed when the amphiphilic compound self-assembles by at least about 20 nm.

8. The tablet according to claim 4, wherein (i) the amphiphilic compound, in the presence of the water channel enhancer, is capable of self-assembling into cubic Im3m liquid crystalline particles and (ii) the amphiphilic compound in the absence of the water channel enhancer is capable of self-assembling into cubic Pn3m liquid crystalline particles.

9. The tablet according to claim 4, wherein the water channel enhancer is selected from the group consisting of Tween 80, sodium cyclamate, saccharin sodium, Poloxamer 188, Poloxamer 407, Mg Trisilicate, SLS, menthol, stearic acid, butylated hydroxyl toluene and sodium bicarbonate.

10. The tablet according to claim 1, wherein when the ODT is administered to the oral mucosa, the ODT facilitates systemic administration of the active ingredient across the oral mucosa.

11. The tablet according to claim 1, wherein when the tablet contacts a hydrophilic solvent the amphiphilic compound self-assembles into liquid crystalline particles.

12. The tablet according to claim 1, wherein the oral disintegrating tablet disintegrates in less than 2 minutes following contact with a hydrophilic solvent.

13. The tablet according to claim 1, wherein the amphiphilic compound has a critical packing parameter (CPP) of >½ and/or a hydrophilic lipophilic balance (HLB) of 0 to <10.

14. The tablet according to claim 1, wherein the amphiphilic compound has the structure of Formula (I):
X−T Formula (I)
wherein
X has at least 2 hydrogen bond forming functional groups and is selected from the group consisting of an ester, ether, anhydride, amide, amine, carbamide, glycerol, biuret, phenyl, pyridine or phosphate; and
T is selected form the group consisting of:
(i) a single C12 to C18 alkyl, alkenyl and alkynyl terminally attached to X optionally comprising:
a. one or more double bonds (preferably cis and at about C7 to C11); or
b. three or more methyl branches (preferably isoprenoid branching);
and
(ii) two C12 to C18 alkyl, alkenyl and alkynyl both terminally attached to X.

15. The tablet according to claim 1, wherein the active ingredient has a log P of −0.5 to 6.4.

16. The tablet according to claim 1, wherein the disintegrant is about 10 to about 50% w/w of the tablet.

17. (canceled)

18. The tablet according to claim 1, wherein the disintegrant is sodium starch glycolate, crosslinked polyvinylpyrrolidone or both.

19. The tablet according to claim 1, wherein the amphiphilic compound is a glycerol monoleate.

20. The tablet according to claim 19, wherein the amphiphilic compound, in the absence of a water channel enhancer, forms cubic Pn3m liquid crystalline particles.

21. The tablet according to claim 1, wherein the amphiphilic compound is 1 to 20% w/w of the tablet and/or the active ingredient is 0.5 to 10% w/w of the tablet.

22-47. (canceled)
